(19) United States
(12) Patent Application Publication
(10) Pub. No.: US 2015/0274605 A1
(43) Pub. Date: Oct. 1, 2015

(54) METHODS

(71) Applicant: INSTITUTE OF FOOD RESEARCH, Norwich (GB)

(72) Inventors: Keith William Waldron, Norwich (GB); Peter Ryden, Norwich (GB); David Russell Wilson, Norwich (GB); Graham Keith Moates, Norwich (GB)

(21) Appl. No.: 14/441,175

(22) PCT Filed: Nov. 7, 2013
(86) PCT No.: PCT/GB2013/052920

§ 371 Date: May 6, 2015

(30) Foreign Application Priority Data
Nov. 8, 2012 (GB) 1220129.9

(51) Int. Cl. C05F 5/00; C12P 7/10; C05D 1/00; C05B 17/00; C05C 11/00

(52) U.S. Cl. CPC C05F 5/008; C05B 17/00; C05C 11/00; C05D 1/00; C12P 7/10

(57) ABSTRACT

The present invention relates to a method of producing one or more sugar for bio-alcohol production, comprising the step of degrading bioorganic matter comprising lignocellulose, to generate one or more sugar from the lignocellulose and a degraded bioorganic residue; characterised in that the method further comprises the step of forming a plant growth medium from the degraded bioorganic residue. The invention further relates to plant growth media obtained by the method of the present invention.

### RELATED APPLICATION DATA

This application is the U.S. National Stage of International Application No. PCT/GB2013/052920 filed Nov. 7, 2013, which claims the benefit of and priority to Great Britain Patent Application No. 1220129.9 filed Nov. 8, 2012. Each of the foregoing applications is hereby incorporated by reference in its entirety for all purposes.

### TECHNICAL FIELD

The present invention relates to a method of producing one or more sugar for bio-alcohol production, comprising the step of degrading bioorganic matter comprising lignocellulose, to generate one or more sugar from the lignocellulose and a degraded bioorganic residue; characterised in that the method further comprises the step of forming a plant growth medium from the degraded bioorganic residue. The invention further relates to plant growth media obtained by the method of the invention.

### BACKGROUND

In Europe, bio-alcohols such as bio-ethanol are generally produced from the sugars present in sugar crops (such as sugar beet and sugar cane) or starch crops (such as wheat, corn and potatoes). Liberating suitable sugars from those starting materials is generally easy to achieve, and the subsequent fermentation of those sugars into alcohol can be performed simply using yeasts. For example, where a starch crop is used, digestion is easily carried out using cheap thermally-stable amylases and the resulting sugar subsequently fermented.

However, conflicts over food supply and land usage have made the production and utilisation of bio-alcohol from food crops a controversial topic. Accordingly, there is increasing social and political pressure to develop “second generation” bio-alcohol production technology, which is capable of generating bio-alcohol from non-food sources and/or waste materials. Significant efforts have been made in the biochemical conversion of non-food lignocellulose feedstocks (such as “energy crops”, cellulosic residues, and lignocellulose-rich waste) into sugars for bioalcohol production, which should reduce food vs. fuel pressures.

Production of bio-alcohol from lignocellulose is therefore an important commercial goal. To date, such processes have involved physical, chemical and enzymatic treatments that degrade lignocellulose to make cellulose and related polysaccharides accessible to enzymes, and then convert the cellulose and related polysaccharides into simpler sugars such as glucose, cellobiose and xylose that can be used in industrial processes. However, because lignocellulose and cellulose are particularly stable biological molecules, the conditions needed to degrade them are not trivial and involve expensive pre-treatments (such as steam explosion) to open up the lignocellulosic structure, and high-cost enzymes to convert cellulose into sugars for bio-alcohol production.

Consequently, the production of sugars and bio-alcohol from lignocellulosic biomass is not yet economically viable. Attempts to improve the cost-effectiveness of the production process have focused on increasingly aggressive treatments which seek to maximise the degradation of the starting material and convert as much of the lignocellulose to sugar as possible. Such approaches result in the formation of sugars and a degraded liquid bioorganic waste residue or slurry that is devoid of any physical structure and contains few remaining nutrients.

### SUMMARY

Against this background, the present inventors have developed an alternative method of degrading bioorganic matter comprising lignocellulose, to form sugars for bio-alcohol production. Unlike previous approaches which seek to maximise the degradation of the bioorganic matter to generate as much sugar as possible, the present inventors have realised that degrading the bioorganic matter to a lesser degree can generate not only sugars, but also a commercially-useful co-product from the degraded bioorganic residue. By limiting the severity of the process conditions and only partially-degrading the bioorganic matter, the nature of the resulting degraded bioorganic residue can be controlled, so that it contains a structural components and nutrients, which make it possible to form a valuable plant growth medium.

It is well-known that the requirement for horticultural growing media has increased rapidly since the 1950's as a result of the growth of the Professional Growers industry including nursery stock, pot plants/herbs, bedding plants etc., and amateur gardening. Sphagnum peat has been used as the main constituent of growing media, and the demand has been met principally by UK peat sources, but also by increased import (30%). UK professional growers utilise approximately 1.2 million cubic metres (m³) peat annually. Sphagnum peat satisfies a range of generic grower requirements. These include air porosity (10% at 1 kPa), water holding capacity (WHC; 30%-65%), low nutrient and nitrogen status (that can be regulated), good re-hydration and drainage characteristics and structural stability. All of these underpin modern water and nutrient management practices.

The current supply of peat is under threat as a result of various EU directives, particularly the Wetland Habitats Directive. In addition, targets to reduce bio-waste (e.g. landfill directive) have encouraged National Government to set aspirational targets for reducing peat use in horticulture (90% by 2010), the hope being that the reduction will be addressed by the use of the alternative media. Major retail chains have declared support for these initiatives, and are pressurising their supply chains accordingly. However, many growers are reluctant to change, due to bad experiences with poorly-formulated peat alternatives produced in the early 1990s.

As discussed in more detail below, the plant growth medium produced from the method of the invention replicates plant-structure-dependent physicochemical (i.e. physical and chemical) characteristics found in high-quality growth media, such as peat. The present method therefore permits the production of sugars for bio-alcohol production and a growing media which is reliable, consistent and predictable for growers in various horticultural sectors. The formation of two valuable types of product further improves the economic viability of processing bioorganic matter comprising lignocellulose.

Thus, in a first aspect the invention provides a method of producing one or more sugar for bio-alcohol production, comprising the step of degrading bioorganic matter comprising lignocellulose to generate one or more sugar from the lignocellulose and a degraded bioorganic residue; characterised in that the method further comprises the step of forming a plant growth medium from the degraded bioorganic residue.

Thus, the invention provides a method of producing one or more sugar, and a plant growth medium, from bioorganic matter comprising lignocellulose. The inventors have developed a method which generates those two types of product by partially degrading the bioorganic matter comprising lignocellulose. Doing so results in the generation of some sugars but additionally leaves a degraded bioorganic residue containing structural components which is suitable for forming into a plant growth medium. The conditions used in the method of the invention therefore require a “balance” between sufficient severity of pre-treatment and enzymatic digestion to generate sugars for fermentation vs. retaining structural components in the residue.

The present method is therefore distinct from prior art approaches which focused on the complete degradation of bioorganic matter to maximise sugar yield, and resulted in a bioorganic residue which was devoid of physical structure and unsuitable for producing plant growth media.

As discussed further below, the plant growth medium is preferably solid or semi-solid and preferably a peat-replacement material—importantly, that plant growth medium comprises structural components which support the formation of plant growth. The plant growth medium is a valuable co-product.

It will be appreciated that sugars generated by the method of the present invention are suitable for a range of uses. For example, such sugars may be used to produce fermentation products such as succinate; itaconic acid; sophorolipid; alcohol (i.e. bio-alcohol); and products of chemical catalysis, such as furfurals. Accordingly, the method of the invention may be used to produce a plant growth medium and a product selected from the group consisting of: succinate; itaconic acid; sophorolipid; alcohol (i.e. bio-alcohol); and furfurals.

Preferably, the method of the invention is used to produce bio-alcohol, and therefore also comprises the step of producing bio-alcohol from the one or more sugar. Accordingly, in a preferred embodiment, the invention relates to a method of producing bio-alcohol comprising the step of degrading bioorganic matter comprising lignocellulose, to generate one or more sugar from the lignocellulose and a degraded bioorganic residue; characterised in that the method further comprises the step of forming a plant growth medium from the degraded bioorganic residue.

As is well known in the art, bio-alcohol is a general term given to alcohol that has been manufactured from bioorganic starting material, and which is typically generated by fermentation. Examples of bio-alcohols include: bio-ethanol; bio-butanol; bio-isobutanol; and bio-acetone.

In a particularly preferred embodiment, the method comprises the following steps:
(a) providing an amount of bioorganic matter comprising lignocellulose;
(b) degrading the bioorganic matter to generate one or more sugar from the lignocellulose and a degraded bioorganic residue; and
(c) forming bio-alcohol from the one or more sugar;
wherein the method further comprises the step, performed after step (b) or after step (c), of forming a plant growth medium from the degraded bioorganic residue.

In a preferred embodiment, steps (b) and (c) are performed simultaneously, for example using simultaneous saccharification and fermentation (SSF), as described below.

As discussed further below, step (b) involves the conversion of lignocellulose in the bioorganic matter into one or more sugar, whilst generating a degraded bioorganic residue that retains sufficient structure to support plant growth and which can subsequently be converted into a plant growth medium. Step (c) involves the conversion of the one or more sugar into bio-alcohol.

The bioorganic matter comprising lignocellulose for use in the method of the invention may be any lignocellulose-containing material. The amount of lignocellulose present in bioorganic matter will vary but a preferred lignocellulose content is approximately 60-90% of lignocellulose by dry weight of the total weight of the bioorganic matter.

Typically, the bioorganic matter comprises plant matter comprising lignocellulose, preferably selected from the group consisting of lignified plant matter and semi-lignified plant matter. Lignified and semi-lignified plant matter includes lignified vascular and related tissues (i.e. “fibres”), and lignified palea and lemma from the outer part of cereal grains (i.e. “sheets”) which are present, for example, in Brewers' grain residues.

More preferably, the lignified plant matter and/or semi-lignified plant matter comprises or consists of sheets and/or fibres of lignified plant matter. Sheets of lignified matter also includes matter derived from wood shavings or other processed wood material.

It is most preferred that plant matter comprising lignocellulose is selected from the group consisting of: wood; wood chippings; straw; straw leaves; cereal leaves; brewer's grain; wheat bran; oat grain; rice bran; grasses (such as Miscanthus species).

It will be understood that the method of the invention can be performed using any amount of bioorganic matter comprising lignocellulose. It is preferred that the amount of bioorganic matter comprising lignocellulose is at least 10 kg, for example at least 20 kg, 30 kg, 40 kg, 50 kg, 60 kg, 70 kg, 80 kg, 90 kg, 100 kg, 150 kg, 200 kg, 250 kg, 300 kg, 400 kg, 500 kg, 10 tonnes, 20 tonnes, 50 tonnes, 100 tonnes, 200 tonnes, 300 tonnes, 500 tonnes, 1,000 tonnes, 2,000 tonnes, 5,000 tonnes, 10,000 tonnes, 20,000 tonnes, 50,000 tonnes, 100,000 tonnes, 200,000 tonnes, 300,000 tonnes, 400,000 tonnes, 500,000 tonnes, 600,000 tonnes, 700,000 tonnes, 800,000 tonnes, 900,000 tonnes, 1,000,000 tonnes or more.

### BRIEF DESCRIPTION OF THE DRAWINGS

FIG. 1—Impact of severity factor on the enzymatic production of glucose, “coarse” media and “fine” media from wheat straw. Key: ▪>1.4 mm; □<1.4 mm; ▴ glucose.

FIG. 2—FTIR spectra of “coarse” and “fine” fractions produced after enzymatic digestion (BioCatalysts Ltd enzymes) of straw that has been pre-treated (by steam explosion) at 195° C. for 10 minutes. The results highlight the lower level of carbohydrate in the “fine” fraction.

FIG. 3—FTIR spectra of “coarse” fractions produced after enzymatic digestion (BioCatalysts Ltd enzymes) of straw that has been pre-treated (by steam explosion) at 180° C. to 230° C. for 10 minutes. The results show that at higher pre-treatment severities, the levels of carbohydrate in the “coarse” material decreases.

FIG. 4—Key physical properties of recalcitrant material resulting from the enzymatic digestion (using BioCatalysts Limited enzymes) of steam-exploded wheat straw. Digestion was performed for 42 hours at 5% substrate concentration, as described in the Examples.

FIG. 5—Principal Components Analysis of “coarse” materials, obtained as in FIG. 3. Principal Components Analysis (“PCA”) is used to compare samples of growing media, peat-based products and recalcitrant residues. Grey-filled circles—general growing media and composted material; white-filled circles—peat-based products; black filled circles—range of recalcitrant coarse materials produced after saccharification of steam-exploded straw as described in FIG. 3. Temperatures of pre-treatment (by steam explosion) are shown next to the black circles. The results show that as intensity of pre-treatment (by steam explosion) increases, the quality of the coarse material moves towards the area frequented by high-quality growing media (dotted line).

FIG. 6A—Germination of lettuce seeds in: commercial compost; “coarse” residue from wheat straw steam-exploded at 210° C. (18.1 bar) for 10 min and digested with Biocatalysts enzymes for 42 h; and a 50:50 mixture of the two.

FIG. 6B—Germination of lettuce seeds in: peat-based growing media; “coarse” residue from wheat straw steam-exploded at 210° C. (18.1 bar) for 10 min and digested with Biocatalysts enzymes for 42 h; and a 50:50 mixture of the two.

FIGS. 7A and 7B—Germination of cucumber seedlings. Cucumber seeds were germinated under controlled conditions on the various residues (coarse recalcitrant residue from wheat straw produced by steam explosion followed by enzymatic digestion) over a 2 day period. Details are shown in Example 1.

FIG. 7A: Visual seedling score from 0 (seedling dead) to 5 (exceptional growth and fullness).

FIG. 7B: % normal cucumber seedlings as assessed by methods of the International Seed Testing Association.

FIG. 8—Cucumber seedlings grown on control and coarse material (from FIG. 7).

FIG. 9—Saccharification of “fines” using multiple additions to increase the final glucose concentration. “Fines” were produced by enzymatic digestion of pre-treated wheat straw (195° C. for 10 min). The enzymes used for production of “fines” were Novozymes Cellic HTec2 for 24 h in a stirred bioreactor at 50° C., pH 5. Details are given in Example 2. The “fines” were then subjected to saccharification with Cellic CTec2 and a 10% substitution with Cellic HTec2 (pH5, 50° C.) at varying substrate and enzyme concentrations. The results show that batch addition of fines and enzymes can increase the concentration of saccharified glucose to approximately 0.5 mol/L.

FIG. 10—Dry matter proportions of coarse □, fines ∘, and glucose Δ obtained from Cellic HTec2 digestions of wheat straw steam-exploded at 190° C. for 10 min.

FIG. 11—High torque bioreactor (15 Litre capacity)

The bioreactor comprises: a heating means that is capable of heating and maintaining the reactants at a temperature suitable for correct functioning of the enzymes (for example, 30-70° C., and preferably 50° C.); and means for mixing the reactants at between 10-65 RPM, the rate of which is dependent on the stiffness of the reactants being mixed.

FIG. 12—A Viola growing trial photographed at approximately 3, 5 and 7 weeks. Wheat straw was steam-exploded and separated into coarse and fines with 1.2% Cellic HTec2. The steam-exploded material before HTec2 treatment and the coarse recalcitrant material were used at 100% and in a 50% blend with a standard multi-purpose compost.
A: Date of photograph Jul. 4, 2013;
B: Date of photograph Jul. 10, 2013;
C: Date of photograph Jul. 24, 2013.
In each case, numbering from the left, the 5 pots contain:
1. 100% Coarse recalcitrant media.
2. 50% Coarse recalcitrant media.
3. Standard multipurpose compost.
4. 100% Steam-exploded straw.
5. 50% steam-exploded straw.

FIG. 13—Over 200 L coarse material produced for growing trials.

FIG. 14—Demonstration of viola plants after several weeks growing in growing media created using the large-scale digestions in Example 4.

FIG. 15—The effect of enzyme concentration on the yield of ethanol through SSF.

FIG. 16—Scheme outlining exemplary methods of the invention.

### DETAILED DESCRIPTION

By “degrading the bioorganic matter” we include the step of reducing the overall level of physical structure in that matter. Importantly, the bioorganic matter is not fully degraded to remove all of its physical structure and/or structural components—instead, it is partially degraded so that the resulting residue retains some structure and/or one or more structural component.

As discussed below, that step may include subjecting the bioorganic matter to physical conditions (such as pressure and/or temperature and/or shear forces) capable of physically breaking down structural and/or rigid components within it; alternatively or additionally, that step may include degradative chemical or enzymatic treatments which remove structural components or convert complex molecules into simple ones.

By “degraded bioorganic residue” we include matter derived from the bioorganic matter and which contains one or more components of the bioorganic matter that has not been degraded (or fully degraded). For example, the residue may contain one or more components of the bioorganic matter that cannot be degraded (or fully degraded) by the method of the invention (for example, because that component is resistant to the physical, biochemical or enzymatic conditions of the method).

Alternatively, or additionally, the degraded bioorganic residue may contain one or more components of the bioorganic matter that has not been degraded (or fully degraded) because the conditions were not sufficient to do so—for example, the conditions may have been maintained at a temperature, or for a time, not permitting the degradation (or full degradation) of that one or more component.

Preferably, the degraded bioorganic residue produced by the method of the invention comprises matter that retains one or more complex chemical components and/or one or more physical structural components of the bioorganic matter. Preferably, the degraded bioorganic residue is solid or substantially solid and comprises rigid and/or substantially rigid components derived from the bioorganic matter so that the resulting plant growth medium has a partially rigid or defined structure (for example, the consistency of peat). In short, the “degraded bioorganic residue” includes partially-degraded bioorganic matter which comprises a structure capable of supporting plant growth.

By “plant growth medium” we include a solid or semi-solid medium capable of promoting and/or increasing plant growth (and/or the germination of seeds, bulbs or tubers thereof) either when used alone or when mixed with other plant growth media, supplements and/or fertilisers to form a complex plant growth medium.

Preferably, the plant growth medium of the invention is a solid or substantially solid medium, which has the consistency of peat. By “solid or substantially solid” we include the meaning that the medium is sufficiently solid to facilitate and support root growth and development, and the growth of aerial organs, either when dry or when saturated with water.

Peat, particularly that derived from sphagnum peat bogs, is known to retain a high level of plant structure, both at the tissue, cell and cell-wall length scales. It is this structure that underpins the balance of functional characteristics prized by growers, such as aeration, water-retention, good drainage and low nutrient content. In a preferred embodiment, the present method is used to produce a plant growth medium which is a peat-substitute material (i.e. a material having characteristics of peat, such as some or all of the biochemical, structural and microbiological characteristics of peat), and which can be used instead of peat in applications in which peat is typically used. Whilst peat has historically been used as a plant growth medium, it may also be used as a fuel (i.e. a solid or substantially-solid fuel) that is burned to generate energy or used to generate a product in which carbon is sequestered, potentially for long-term carbon storage.

It will be appreciated that the method of the invention may be used to make plant growth media having other defined characteristics, compositions and/or consistencies, as desired. For example, the method of the invention may be used to produce growth media ranging from relatively dry growing media through to wet media suitable for hydroponic uses, as are known in the art. Thus, the plant growth medium produced by the method of the invention may range from relatively “fluid” material with small particle sizes through to more entangled material with fibres of 1 to 10 cm in length (for use, for example, in robotic plant handling and propagation systems).

Preferably, the plant growth medium of the invention comprises rigid or substantially rigid components derived from the bioorganic matter so that the plant growth medium has a partially rigid or defined structure. For example, the plant growth medium may comprise structural components such as: lignified and semi-lignified plant matter; residual plant cell-wall material enriched in non-carbohydrate components such as lignin, protein, waxes and lipids, and inorganic salts.

Typically, following completion of step (b) of the method of the invention, the degraded bioorganic residue comprises particles of various shapes and sizes. Optionally, after step (b) but before the step of forming a plant growth medium, the larger particles (termed the “coarse” fraction) may be separated from the smaller particles (termed the “fine” fraction), for example, by sieving or other known wet or dry particle-separation approaches (such as draining, centrifugation, and/or spin-drying).

A particularly preferred small-scale separation approach is to perform sieving on nylon bolting cloth (1.32 mm pore size) with low-speed centrifugation using spin-drying. The separated coarse material typically contains 2-4 grams of water per gram of dry weight and can be air-dried prior to performing subsequent steps.

If such a separation is performed, it is preferred that the “coarse” fraction (which comprises lignified cell wall material and, possibly, silica and silicate) is used to form a plant growth medium, whilst the “fine” fraction is subjected to additional rounds of degradative treatments which generate further sugars from any lignocellulose and/or polysaccharides remaining.

Typically, the “coarse” fraction consists of particles which have at least one dimension of 1.32 mm or more (such as 1.50 mm, or 2.00 mm; or 2.50 mm; or 3.00 mm; or 3.50 mm; or 4.00 mm; or 4.50 mm; or 5.00 mm; or 5.50 mm; or 6.00 mm; or 6.50 mm; or 7.00 mm; or 7.50 mm; or 8.00 mm; or 8.50 mm; or 9.00 mm; or 9.50 mm; or 10.00 mm; or 20.00 mm; or 50.00 mm; or 100.00 mm, or greater).

Typically, the “fine” fraction consists of particles which have at least one dimension of less than 1.32 mm (such as 1.00 mm, or 0.90 mm; or 0.80 mm; or 0.70 mm; or 0.60 mm; or 0.50 mm; or 0.40 mm; or 0.30 mm; or 0.20 mm; or 0.10 mm or less).

Preferably, step (b) of the method (which involves degrading the bioorganic matter) comprises the step of: (b-i) subjecting the bioorganic matter to conditions capable of melting and/or hydrolysing and/or solubilising some or all of the lignocellulose in the bioorganic matter.

As is known in the art, a range of physical and chemical conditions (such as thermophysical and thermochemical conditions) can be used to melt and/or solubilise lignocellulose, and doing so typically degrades the lignin component of lignocellulose, and liberates cellulose and other polysaccharides. The cellulose and other polysaccharides are rendered more readily-available for conversion, and can subsequently be converted into sugars using methods known in the art, such as enzymatic conversion.

Once generated, the one or more sugars may be fermented to form bio-alcohol. Fermentation may be performed by contacting the one or more sugar with one or more microbial agent, as are known in the art (see, for example, Waldron (ed.) Bio-alcohol Production: Biochemical Conversion of Lignocellulosic Biomass, 2010; Woodhead Publishing Limited). For example, microorganisms (including yeasts, such as Saccharomyces species, and bacteria, such as Clostridia species) are commonly used to form bio-ethanol by the fermentation of glucose and/or pentose sugars, typically in a bioreactor and by known methods such as “SSF” or “SSSF” (“simultaneous saccharification and fermentation” or “semi-simultaneous saccharification and fermentation”, respectively).

Lignocellulose can be melted and/or hydrolysed and/or solubilised by subjecting it to high temperature and/or pressure and/or humidity, which is thought to rehydrate lignocellulose and physically and/or chemically degrade the rehydrated fibres and cells. Other approaches are also known to be suitable, including treatments using alkaline or acidic solutions (such as treatments involving formic acid and/or acetic acid).

Preferably, the conditions used in step (b-i) of the method comprise heating at a temperature of between approximately 100° C. and approximately 240° C., preferably at a temperature of between approximately 190° C. and approximately 240° C. Particularly preferred conditions comprise heating at a temperature of: approximately 100° C.; or approximately 110° C.; approximately 120° C.; or approximately 130° C.; or approximately 140° C.; or approximately 150° C.; or approximately 160° C.; or approximately 170° C.; or approximately 190° C.; or approximately 190° C.; or approximately 200° C.; or approximately 210° C.; or approximately 220° C.; or approximately 230° C.; or approximately 240° C. Heating at such temperatures is typically performed for between 1 and 20 minutes, and preferably for 20 minutes, and more preferably for 10 minutes.

Preferably, the conditions used in step (b-i) of the method comprise heating (as defined above) at a pressure of between 7 bar to 27 bar. Pressurised reactors suitable for performing heating at such pressures are known in the art.

Preferably, the conditions used in step (b-i) of the method comprise heating (as defined above) the bioorganic matter under high humidity and/or in the presence of water or moisture; for example, the bioorganic matter may be provided in an aqueous form. Doing so avoids the bioorganic matter becoming scorched or burned during heating. Preferably, heating the bioorganic matter under high humidity and/or in the presence of water or moisture is performed in a pressurised reactor, as are known in the art.

Particularly preferred conditions used in step (b-i) comprise steam applied for between 5 and 15 minutes at a temperature between 170° C. to 240° C.

It is well known that the severity of a particular treatment is determined by the temperature (T) and time (t) of that treatment, which can be expressed as a “Severity Factor”, calculated using the following equation:

Severity Factor=log₁₀(t×exp((T−100° C.)/14.75))

The inventors have found that, within the method of the present invention, treatments providing a Severity Factor of between 3.6 to 4.0 are particularly preferred and generate a degraded bioorganic residue which comprises sufficient structural components to permit production of plant growth medium.

The severity of the conditions that need to be used in step (b-i) will be determined partly by the particular type of bioorganic matter being treated. Some bioorganic matter will require relatively mild treatments (i.e. relatively low temperatures maintained for relatively short times) in order to melt and/or hydrolyse and/or solubilise the lignocellulose—for example, straw that has been allowed to partially degrade microbially through storage outside over the winter period may require a milder treatment than fresh straw or wood chippings, and there may also be tissue- and varietal-differences in substrate quality as known in the art.

Those skilled in the art would be aware of methods for determining the extent of degradation in order to assess whether conditions are of appropriate severity. For example, the degraded bioorganic residue generated by a particular treatment can be analysed to determine the amount of soluble components derived from lignocellulose and/or the structural components remaining.

In a preferred embodiment, step (b-i) of the method of the invention is performed using steam explosion.

Steam explosion is a well known process which involves injecting steam into a sealed vessel until a desired pressure is obtained, before suddenly releasing the pressure and diverting the expelled matter through a cyclone to separate steam from liquid and solids (see, for example, Overend & Chornet (1987; Philos. T. R. Soc. A., 321: 523-536) and Wi et al., (2011; Bioresource Technology, 102: 5788-579).

Alternatively, step (b-i) is performed using one or more method selected from: hot-water treatment; AFEX (Ammonia Fibre Explosion or Ammonia Fibre Expansion); extrusion; autoclaving. Such methods are well known in the art.

AFEX is performed using a similar process to steam explosion, but involves treatment with liquid anhydrous ammonia at a temperature of 60-100° C. and at high pressure (typically 250-300 psi; 17.2-20.7 bar) for 5 minutes, before sudden pressure release.

Extrusion involves immersion of the matter to be treated in water (with or without alkali, such as sodium hydroxide at a concentration of 0.1-10%; or with or without acids, such as acetic acid at a concentration of 0.01-5%), followed by agitation by rotation at 50-200 rpm at a temperature of 20-250° C.

Autoclaving is a well known process which involves injecting steam at high pressure into a sealed vessel and heating at high temperature and pressure (such as 121° C. for 15 minutes at 1 bar; or 135° C. for 3 minutes at 2.1 bar).

Preferably, step (b) of the method (which involves degrading the bioorganic matter) further comprises the steps, performed after step (b-i), of:
(b-ii) optionally, washing the treated bioorganic matter;
(b-iii) subjecting the treated bioorganic matter to conditions capable of degrading plant cell walls in the bioorganic matter.

Step (b-ii) may be performed by forming a slurry of the treated bioorganic matter in water (preferably warm water, for example at 50° C.) and then removing the water (for example, by sieving; draining; centrifugation; and/or spin-drying).

Step (b-ii) removes inhibitors (such as enzyme inhibitors and/or fermentation inhibitors) that would prevent or reduce the subsequent fermentation step—for example, compounds that are toxic to yeasts and/or fermentative enzymes and compounds that inhibit the action of cellulases and/or hemicellulases (see, for example, Garcia-Aparicio et al., Chemistry and Materials Science: 27th Symposium on Biotechnology for Fuels and Chemicals ABAB Symposium, 2006, Session 1B, 278-288, DOI: 10.1007/978-1-59745-268-7_{—}22). Such inhibitors include: formic acid; acetic acid; levulinic acid; furfural; 5-hydroxymethyl furfural; syringic acid; 4-hydroxy benzaldehyde; and vanillin. Step (b-ii) may also remove inhibitors that could reduce or prevent plant growth and/or germination, and which could be detrimental in the resulting plant growth medium.

It is preferred that the conditions in step (b-iii) of the method comprise contacting the bioorganic matter with one or more enzyme selected from the group consisting of: a cellulase; a hemicellulase; a pectinase; an esterase; a protease; a xylanase; an oxido-hydrolase. The bioorganic matter can be contacted either with a preparation or composition of the one or more enzyme, or with one or more microorganisms expressing the one or more enzyme. Advantageously, the one or more enzyme (and/or the microorganism expressing the one or more enzyme) is thermophilic or mesophilic.

In a preferred embodiment, step (b-iii) of the method comprises mixing and/or agitating the contacted bioorganic matter and one or more enzyme.

Typically, treatment with one or more enzymes has the result of further fractionating the bioorganic matter to provide a “coarse” fraction and “fine” fraction, as discussed above.

Cellulose is a homopolymer of glucose and is usually the most abundant polysaccharide in agricultural waste. It forms partially-crystalline rods and is hydrolysed by cellulases.

Hemicelluloses are heteropolymers, commonly comprising the monosaccharides: xylose, arabinose, mannose, galactose, glucose and/or glucuronic acid. These polysaccharides can be partially acetylated and they are covalently linked to phenolics and lignin, and have a cross-linking function in the cell wall. Many enzymes are needed to digest hemicelluloses, including: arabinases; α-L-arabinofuranosidase; acetyl xylan esterase; β-mannanase; glucuronidase; β-xylanase.

Esterases for use in step (b-iii) of the method include; acetyl xylan esterase (which removes acetyl groups for xylan); ferulic acid esterase (which removes ferulic acid from hemicelluloses). Oxido-hydrolases for use in step (b-iii) of the method catalyse the breaking of sugar linkages and oxidize the reducing end of the sugars.

Where step (b-iii) involves a preparation or composition of one or more enzyme, suitable enzymes and compositions may be obtained from companies such as Biocatalysts (UK), Novozymes (Denmark), and DuPont Genencor (US).

Typically, step (b-iii) comprises a pH of between 4.5 and 5. If the treated bioorganic matter is not at a suitable pH prior to step (b-iii), the pH may be adjusted using an appropriate buffer, such as sodium acetate (typically, at a buffer concentration of 50 mM) or non-organic phosphate buffer.

Conveniently, the one or more sugar generated from the lignocellulose is glucose and/or cellobiose and/or xylose and/or arabinose and/or mannose and/or galactose and/or glucuronic acid and/or galacturonic acid and/or fucose and/or rhamnose. It will be appreciated that the one or more sugar generated is dependent on the specificity of the one or more enzyme used in the method of the invention.

Once the one or more sugar has been generated, further steps are performed to form a plant growth medium from the degraded bioorganic residue and form bio-alcohol from the one or more sugars. The plant growth medium may be formed: by separate production of the plant growth medium and bio-alcohol; or by partially-separated production of the plant growth medium and bio-alcohol; or by combined production of the plant growth medium and bio-alcohol. Those three embodiments of the invention are discussed below.

### 1. Separate Production of a Plant Growth Medium and Bio-Alcohol

In a particular embodiment of the invention, the method comprises separating the degraded bioorganic residue from the one or more sugar. Separate processes can then be performed on the separated materials to generate a plant growth medium and bio-alcohol—particularly, as discussed below, the degraded bioorganic residue is used to produce a plant growth medium, and the one or more sugar is used to produce bio-alcohol.

In that embodiment of the invention, the method further comprises the step, performed after step (b), but before step (c), of: (b′) separating the degraded bioorganic residue from the one or more sugar generated from the lignocellulose.

Typically the one or more sugar generated from the lignocellulose is present in a liquid phase, whilst the degraded bioorganic residue comprises solid matter at a range of particle sizes. Steps suitable for separating such solids from liquids are well known in the art and can therefore be used to fully or partially separate the degraded bioorganic residue from the one or more sugar—for example, centrifugation, belt-press dewatering, and/or sifting can be used.

As discussed above, the degraded bioorganic residue comprises particles of various shapes and sizes and the larger particles in that residue (termed the “coarse” fraction) may be separated from the smaller particles in that residue (termed the “fine” fraction), for example, by sieving or other known particle-separation approaches. Such a separation may be performed at the same time as, or immediately after, step (b′). If such a separation is performed, it is preferred that the “coarse” fraction is used to form a plant growth medium, whilst the “fine” fraction is subjected to additional rounds of degradative treatments which generate further sugars from any lignocellulose and/or polysaccharides remaining.

Preferably, forming a plant growth medium from the degraded bioorganic residue comprises the steps, performed after step (b′), but before step (c), of:
(x-i) providing the degraded bioorganic residue, generated by step (b′);
(x-ii) washing the degraded bioorganic residue;
(x-iii) optionally, subjecting the degraded bioorganic residue to conditions capable of decomposing the bioorganic residue, and inhibiting decomposition prior to its completion;
(x-iv) removing moisture from the resulting degraded bioorganic residue.

The degraded bioorganic residue provided in step (x-i) may comprise the “coarse” fraction which has been separated from the “fine” fraction. Alternatively, the degraded bioorganic residue provided in step (x-i) may not have been subjected to a size separation step and therefore comprise both “coarse” and “fine” fractions.

Step (x-ii) may be performed by forming a slurry of the treated bioorganic matter in water (preferably warm water, for example at 50° C.) and then removing the water (for example, by sieving; draining; centrifugation; and/or spin-drying). That washing step removes nutrient sources, microbial agents and/or enzymes which are not desired in the resulting plant growth medium.

Preferably, the plant growth medium produced by the method of the invention is stable, insofar as no detectable decomposition of that medium occurs over time. One way of ensuring that no detectable decomposition occurs is to produce a plant growth medium in which there is no (or substantially no) microbial biomass and/or microbial nutrients from the plant growth medium, which can be achieved by washing the degraded bioorganic residue to remove all (or substantially all) microbial biomass and microbial nutrients.

It will be understood that relatively small amounts of microorganisms, degrading enzymes and nutrient sources will be tolerated in the plant growth medium. Furthermore, small quantities of microbial biomass (including, for example, associated extracellular polymers) may be beneficial in the plant growth medium, as it may provide advantageous properties (such as, for example, water retention). Preferably, the amounts of microorganisms, degrading enzymes and nutrient sources in the plant growth medium are not sufficient to begin or allow further decomposition of the plant growth medium.

The resulting washed degraded bioorganic residue can be analysed to determine whether sufficient microbial biomass and/or microbial nutrients have been removed. The microbial nutrients comprise or consist of readily-digestible insoluble starch and/or protein and/or lipid and/or partially-saccharified cell-wall structuring material. Methods for evaluating starch and/or protein and/or lipid and/or bio-available cell-wall-derived sugars (such as rhamnose, fucose, arabinose, xylose, mannose, galactose and glucose) are well-known to those skilled in the arts of chemistry and biochemistry. The level of microbial stability may be determined using, for example, the Solvita test which evaluates ammonia generation which is available as a commercial kit from Solvita (Coventry, UK).

Optionally, the degraded bioorganic residue may be subjected to further decomposition steps to improve its structure and suitability as a plant growth medium. Such methods are described in WO 2008/084210.

Step (x-iv) may be performed to remove moisture from the treated residue so that the resulting plant growth medium is solid or is substantially-solid. Any method capable of removing moisture from aqueous solution or semi-solid matter which are known in the art may be used, such as: dewatering; centrifugation; pressing; or sifting. In practice, a pressing system (such as a belt-pressing system for dewatering) is preferred, as is known in the art. Conveniently, the moisture content of the residue following sub-step (x-iv) is approximately 15-75%.

In that embodiment of the invention, step (c) comprises the steps of:
(c-1) providing the one or more sugar generated from lignocellulose, generated by step (b′); and
(c-2) forming bio-alcohol from the one or more sugar by fermentation;
(c-3) optionally, separating the bio-alcohol from the fermentate.

It will be appreciate that step (c-3) may be performed by methods known in the art, such as distillation, pervaporation and/or membrane filtration.

Preferably, fermentation is performed by contacting the one or more sugar with one or more microbial agent, as are known in the art (see, for example, Waldron (ed.) Bio-alcohol Production: Biochemical Conversion of Lignocellulosic Biomass, 2010; Woodhead Publishing Limited). For example, microorganisms (including yeasts, such as Saccharomyces species; and bacteria, such as Clostridia species) are commonly used to form bio-ethanol by the fermentation of glucose and/or pentose sugars, typically in a bioreactor and by known methods such as “SSF” or “SSSF” (“simultaneous saccharification and fermentation” or “semi-simultaneous saccharification and fermentation”, respectively).

Thus, in a particularly preferred embodiment of the invention, the method comprises the steps:
(a) providing an amount of bioorganic matter comprising lignocellulose;
(b) degrading the bioorganic matter to generate one or more sugar from the lignocellulose and a degraded bioorganic residue, by:
   (b-i) subjecting the bioorganic matter to conditions capable of melting and/or hydrolysing and/or solubilising some or all of the lignocellulose in the bioorganic matter.
   (b-ii) optionally, washing the treated bioorganic matter; and
   (b-iii) subjecting the treated bioorganic matter to conditions capable of degrading plant cell walls in the bioorganic matter;
(b′) separating the degraded bioorganic residue from the one or more sugar generated from the lignocellulose;
(c) forming bio-alcohol from the one or more sugar, by:
   (c-1) providing the one or more sugar generated from lignocellulose, generated by step (b′); and
(c-2) forming bio-alcohol from the one or more sugar by fermentation;
(c-3) optionally, separating the bio-alcohol from the fermentate.
wherein the method further comprises the step, performed after step (b) or after step (c), of forming a plant growth medium from the degraded bioorganic residue, by:
   (x-i) providing the degraded bioorganic residue generated by step (b′); or, providing the coarse fraction of the degraded bioorganic residue generated by step (b′);
   (x-ii) washing the degraded bioorganic residue;
   (x-iii) optionally, subjecting the degraded bioorganic residue to conditions capable of decomposing the bioorganic residue, and inhibiting decomposition prior to its completion; and
   (x-iv) removing moisture from the resulting degraded bioorganic residue.

### 2. Partially-Separated Production of a Plant Growth Medium and Bio-Alcohol

In an alternative particular embodiment of the invention, the method comprises separating the “fine” fraction of the degraded bioorganic residue and the one or more sugar from the “coarse” fraction of the degraded bioorganic residue. Separate processes can then be performed on the separated materials to generate a plant growth medium and bio-alcohol—particularly, as discussed below, the “coarse” fraction of the degraded bioorganic residue is used to produce a plant growth medium, whilst bio-alcohol is produced from the mixture of the “fine” fraction of the degraded bioorganic matter and the one or more sugar.

In that embodiment of the invention, the method further comprises the step, performed after step (b), but before step (c), of: (b″) separating the “coarse” fraction of the degraded bioorganic residue from the “fine” fraction of the degraded bioorganic residues and the one or more sugar generated from the lignocellulose.

Typically the one or more sugar generated from the lignocellulose is present in a liquid phase, whilst the degraded bioorganic residue comprises solid matter at a range of particle sizes. When mixed or agitated, the “fine” fraction is dispersed in the liquid phase. Steps suitable for separating solids from liquids are well known in the art and can therefore be used to separate the “coarse” fraction of the degraded bioorganic residue from the “fine” fraction of the degraded bioorganic residue and the one or more sugar—for example, centrifugation, belt-press dewatering, and/or sifting can be used.

Preferably, forming a plant growth medium from the degraded bioorganic residue comprises the steps, performed after step (b″), but before step (c), of:
(y-i) providing the “coarse” fraction of the degraded bioorganic residue, generated by step (b″);
(y-ii) washing the “coarse” fraction of the degraded bioorganic residue;
(y-iii) optionally, subjecting the “coarse” fraction of the degraded bioorganic residue to conditions capable of decomposing the bioorganic residue, and inhibiting decomposition prior to its completion;
(y-iv) removing moisture from the resulting degraded bioorganic residue.

Step (y-ii) may be performed by forming a slurry of the treated bioorganic matter in water (preferably warm water, for example at 50° C.) and then removing the water (for example, by sieving; draining; centrifugation; and/or spin-drying). That washing step removes nutrient sources, microbial agents and/or enzymes which are not desired in the resulting plant growth medium.

Preferably, the plant growth medium produced by the method of the invention is stable, insofar as no detectable decomposition of that medium occurs over time. One way of ensuring that no detectable decomposition occurs is to produce a plant growth medium in which there is no (or substantially no) microbial biomass and/or microbial nutrients from the plant growth medium, which can be achieved by washing the degraded bioorganic residue to remove all (or substantially all) microbial biomass and microbial nutrients.

It will be understood that relatively small amounts of microorganisms, degrading enzymes and nutrient sources will be tolerated in the plant growth medium. Furthermore, small quantities of microbial biomass (including, for example, associated extracellular polymers) may be beneficial in the plant growth medium, as it may provide advantageous properties (such as, for example, water retention). Preferably, the amounts of microorganisms, degrading enzymes and nutrient sources in the plant growth medium are not sufficient to begin or allow further decomposition of the plant growth medium.

The resulting washed degraded bioorganic residue can be analysed to determine whether sufficient microbial biomass and/or microbial nutrients have been removed. The microbial nutrients comprise or consist of readily-digestible insoluble starch and/or protein and/or lipid and/or partially-saccharified cell-wall structuring material. Methods for evaluating starch and/or protein and/or lipid and/or bio-available cell-wall-derived sugars (such as rhamnose, fucose, arabinose, xylose, mannose, galactose and glucose) are well-known to those skilled in the arts of chemistry and biochemistry. The level of microbial stability may be determined using, for example, the Solvita test which evaluates ammonia generation which is available as a commercial kit from Solvita (Coventry, UK).

Optionally, the degraded bioorganic residue may be subjected to further decomposition steps to improve its structure and suitability as a plant growth medium. Such methods are described in WO 2008/084210.

Step (y-iv) may be performed to remove moisture from the treated residue so that the resulting plant growth medium is solid or is substantially-solid. Any method capable of removing moisture from aqueous solution or semi-solid matter which are known in the art may be used, such as: dewatering; centrifugation; pressing; or sifting. In practice, a pressing system (such as a belt-pressing system for dewatering) is preferred, as is known in the art. Conveniently, the moisture content of the residue following sub-step (y-iv) is approximately 15-75%.

In that embodiment of the invention, step (c) comprises the steps of:
(c-1′) providing the “fine” fraction of the degraded bioorganic residues and the one or more sugar generated from the lignocellulose, generated by step (b″); and
(c-2′) forming bio-alcohol from the “fine” fraction of the degraded bioorganic residues and the one or more sugar by fermentation, preferably by SSF or SSSF;
(c-3′) optionally, separating the bio-alcohol from the fermentate.

It will be appreciate that step (c-3′) may be performed by methods known in the art, such as distillation, pervaporation and/or membrane filtration.

Preferably, fermentation is performed by contacting the one or more sugar with one or more microbial agent, as are known in the art (see, for example, Waldron (ed.) Bio-alcohol Production: Biochemical Conversion of Lignocellulosic Biomass, 2010; Woodhead Publishing Limited). For example, microorganisms (including yeasts, such as Saccharomyces species; and bacteria, such as Clostridia species) are commonly used to form bio-ethanol by the fermentation of glucose and/or pentose sugars, typically in a bioreactor and by known methods such as “SSF” or “SSSF” (“simultaneous saccharification and fermentation” or “semi-simultaneous saccharification and fermentation”, respectively).

Thus, in a particularly preferred embodiment of the invention, the method comprises the steps:
(a) providing an amount of bioorganic matter comprising lignocellulose;
(b) degrading the bioorganic matter to generate one or more sugar from the lignocellulose and a degraded bioorganic residue, by:
   (b-i) subjecting the bioorganic matter to conditions capable of melting and/or hydrolysing and/or solubilising some or all of the lignocellulose in the bioorganic matter.
   (b-ii) optionally, washing the treated bioorganic matter; and
   (b-iii) subjecting the treated bioorganic matter to conditions capable of degrading plant cell walls in the bioorganic matter;
(b″) separating the “coarse” fraction of the degraded bioorganic residue from the “fine” fraction of the degraded bioorganic residue and the one or more sugar generated from the lignocellulose;
(c) forming bio-alcohol from the one or more sugar, by:
   (c-1′) providing the “fine” fraction of the degraded bioorganic residues and the one or more sugar generated from the lignocellulose, generated by step (b″); and
   (c-2′) forming bio-alcohol from the “fine” fraction of the degraded bioorganic residues and the one or more sugar by fermentation, preferably by SSF or SSSF;
   (c-3′) optionally, separating the bio-alcohol from the fermentate;
wherein the method further comprises the step, performed after step (b) or after step (c), of forming a plant growth medium from the degraded bioorganic residue, by:
   (y-i) providing the degraded bioorganic residue generated by step (b″); or, providing the coarse fraction of the degraded bioorganic residue generated by step (b″);
   (y-ii) washing the degraded bioorganic residue;
   (y-iii) optionally, subjecting the degraded bioorganic residue to conditions capable of decomposing the bioorganic residue, and inhibiting decomposition prior to its completion; and
   (y-iv) removing moisture from the resulting degraded bioorganic residue.

### 3. Combined Production of a Plant Growth Medium and Bio-Alcohol

In an alternative embodiment of the invention, the method comprises forming bio-alcohol from the one or more sugar whilst it is still in the presence of the degraded bioorganic residue.

In that embodiment of the invention, fermentation (for example, fermentation by SSF or SSSF) may be performed directly on the product of step (b), which comprises the degraded bioorganic residue and the one or more sugar.

In that embodiment, step (c) comprises the steps of:
(c-1″) providing the degraded bioorganic residue, and the one or more sugar from the lignocellulose;
(c-2″) forming bio-alcohol from the degraded bioorganic residue and the one or more sugar from the lignocellulose, by fermentation;
(c-3″) optionally, separating the bio-alcohol from the degraded bioorganic residue.

As discussed above, fermentation may be performed by contacting the one or more sugar with one or more microbial agent, by methods that are well known in the art. For example, microorganisms (including yeasts, such as Saccharomyces species; and bacteria, such as Clostridia species) are commonly used to form bio-ethanol by the fermentation of glucose and/or pentose sugars, typically in a bioreactor and by known methods such as “SSF” or “SSSF” (“simultaneous saccharification and fermentation” or “semi-simultaneous saccharification and fermentation”, respectively).

Preferably, the method comprises the step, performed after (c-2″), of: (c-3″) separating the bio-alcohol from the degraded bioorganic residue. Processes suitable for separating an alcohol from an aqueous solution are well known in the art, and exemplary methods suitable for performing step (c-3″) of the method of the invention include: filtration (such as vacuum filtration); distillation; reverse osmosis; and partitioning the bio-alcohol to the organic phase.

Preferably, forming a plant growth medium from the degraded bioorganic residue comprises the steps, performed after step (c-3″), of:
(z-i) providing the degraded bioorganic residue, generated by step (c-3″);
(z-ii) washing the degraded bioorganic residue;
(z-iii) optionally, subjecting the degraded bioorganic residue to conditions capable of decomposing the bioorganic residue, and inhibiting decomposition prior to its completion;
(z-iv) removing moisture from the resulting degraded bioorganic residue.

The degraded bioorganic residue provided in step (z-i) may comprise the “coarse” fraction which has been separated from the “fine” fraction. Alternatively, the degraded bioorganic residue provided in step (z-i) may not have been subjected to a size separation step and therefore comprise both “coarse” and “fine” fractions.

Step (z-ii) removes yeast cells and the “fine” fraction and non-fibre components from the degraded bioorganic residues which are not desired in the plant growth medium. That step may be performed by forming a slurry of the treated bioorganic matter in water (preferably warm water, for example at 50° C.) and then removing the water (for example, by sieving; draining; centrifugation; and/or spin-drying). Accordingly, the degraded bioorganic residue remaining after step (z-ii) will comprise or consist of the “coarse” fraction of the degraded bioorganic residue.

As discussed above, the plant growth medium produced by the method of the invention is preferably stable, insofar as no detectable decomposition of that medium occurs over time. One way of ensuring that no detectable decomposition occurs is to produce a plant growth medium in which there is no (or substantially no) microbial biomass and/or microbial nutrients from the plant growth medium, which can be achieved by washing the degraded bioorganic residue to remove all (or substantially all) microbial biomass and microbial nutrients, as discussed above.

Optionally, the degraded bioorganic residue may be subjected to further decomposition steps to improve its structure and suitability as a plant growth medium. Such methods are described in WO 2008/084210.

Step (z-iv) may be performed to remove moisture from the treated residue so that the resulting plant growth medium is solid or is substantially-solid. Any method capable of removing moisture from aqueous solution or semi-solid matter which are known in the art may be used, such as: dewatering; centrifugation; pressing; or sifting. In practice, a pressing system (such as a belt-pressing system for dewatering) is preferred, as is known in the art. Conveniently, the moisture content of the residue following sub-step (z-iv) is approximately 15-75%.

Thus, in a particularly preferred embodiment of the invention, the method comprises the steps:
(a) providing an amount of bioorganic matter comprising lignocellulose;
(b) degrading the bioorganic matter to generate one or more sugar from the lignocellulose and a degraded bioorganic residue, by:
   (b-i) subjecting the bioorganic matter to conditions capable of melting and/or hydrolysing and/or solubilising some or all of the lignocellulose in the bioorganic matter.
   (b-ii) optionally, washing the treated bioorganic matter; and
   (b-iii) subjecting the treated bioorganic matter to conditions capable of degrading plant cell walls in the bioorganic matter;
(c) forming bio-alcohol from the degraded bioorganic residue and the one or more sugar, by:
   (c-1″) providing the degraded bioorganic residue and the one or more sugar from the lignocellulose;
   (c-2″) forming bio-alcohol from the degraded bioorganic residue and the one or more sugar from the lignocellulose, by fermentation; and
   (c-3″) separating the bio-alcohol from the degraded bioorganic residue;
wherein the method further comprises the step, performed after step (c-3′) of forming a plant growth medium from the degraded bioorganic residue, by:
   (z-i) providing the degraded bioorganic residue, generated by step (c-3″);
   (z-ii) washing the degraded bioorganic residue;
   (z-iii) optionally, subjecting the degraded bioorganic residue to conditions capable of decomposing the bioorganic residue, and inhibiting decomposition prior to its completion; and
   (z-iv) removing moisture from the resulting degraded bioorganic residue.

Once a plant growth medium has been produced by the method of the invention, it may be supplemented with additional nutrients or components required in commercial plant growth media, as are known in the art.

For example, a slow-release fertiliser may be added to the plant growth medium produced in step (x-iv) or (y-iv) or (z-iv), and a slow-release fertiliser comprising or consisting of potassium and/or nitrogen and/or phosphorus are particularly preferred. It will be appreciated that other minerals may be added according to the requirements of the plants to be grown in the resulting plant growth medium, and which will be known to those skilled in the art.

Preferably, the method further comprises packaging the plant growth medium. Packaging may be necessary in order to store and transport the plant growth medium, and to present the plant growth medium as a product to horticultural retailers, horticultural growers and other consumers.

The precise nature of the packaging, and the size of each packaged unit, will depend on the particular end consumer. A packaged unit may be a plastic bag of 20-100 litres in size (which are typically provided to horticultural retailers for sale to the public), or larger bags of multi-cubic metre dimensions (which are more appropriate for horticultural growers).

In an embodiment, the method of the invention further comprises the step of analysing a sample of the degraded bioorganic residue, or a sample of the plant growth medium, to determine its physical and/or structural characteristics. Such analysis allows the progress of the method of the invention to be monitored and determine whether the particular treatment conditions are suitable for producing a plant growth medium.

It will be understood that any method capable of monitoring the level or extent of degradation in a sample of the degraded bioorganic residue or plant growth medium could be used). The level or extent of degradation could be determined by analysing the chemical composition and/or physical structure of the residue or medium, for example one or more components that act as a marker of degradation. For example, during degradation one or more nutritional and/or structural components of the bioorganic matter will be altered and/or degraded by the treatment conditions, resulting in a reduction in the amount or concentration of that one or more components which may be used to assess the level or extent of degradation.

Methods for determining the amount or concentration of the one or more components may vary depending on the identity of the component, and suitable methods will be known to those skilled in the art—for example, remote sensing spectroscopy (for example, Fourier transform infra-red spectroscopy (FTIR), near infra-red reflectance (NIR) and nuclear magnetic resonance spectroscopy (NMR) and analytical assays.

Preferably, the level or extent of degradation is determined by analysing the level of structure present in the degraded bioorganic residue which can be performed, for example, by comparative assessment with other growing media using the methods described in WO 2008/084210 and the accompanying Examples.

Preferably, the plant growth medium generated by the method of the invention exhibits one or more of the following properties:
i) no detectable decomposition or minimal detectable decomposition;
ii) a moisture retention of 55% or more at 0.1 bar; for example, 60% or 70% or 80% or 90% or more;
iii) pH6.5 or less; for example, pH6, pH5, pH4, pH3, pH2, pH1 or less;
iv) an electrical conductivity of 422 mS/m or less; for example, 400 mS/m, 300 mS/m, 200 m/m, 100 mS/m, 50 mS/m, 10 mS/m or less;
v) a dry bulk density value of 50 g/L or more, for example, 80 g/L, 100 g/L, 150 g/L, 200 g/L, 250 g/L, 300 g/L, 400 g/L, 500 g/L, 600 g/L or more;
vi) a lignin content of 40% or more; for example, 50%, 60%, 70%, 80%, 90% or more;
vii) an air-filled porosity value of less than 40%, for example, 30%, 27.9%, 25%, 20%, 10%, 5% or less.

More preferably, the plant growth medium generated by the method of the invention exhibits the following properties:
i) no detectable decomposition;
ii) a moisture retention of 60-75% at 0.1 bar;
iii) pH 4.43;
iv) an electrical conductivity of 67 mS/m;
v) a dry bulk density value of 50-110 g/L, and preferably 80-110 g/L;
vi) a lignin content of 40%;
vii) an air-filled porosity value of 10-30%.

By “no detectable decomposition” we include the meaning that no decomposition can be detected over a period of two or three days using any of the tests for monitoring or detecting decomposition described herein—for example, no ammonia production can be detected over a period of two to three days. By “minimal detectable decomposition” we include the meaning that decomposition can be detected using any of the tests described herein but at extremely low rates of decomposition, such as over a period of years, as is found in peat (which is essentially stable, but is still subject to minimal decomposition albeit over an extremely long geophysical period).

Stability may be determined using, for example, the Solvita test which evaluates ammonia generation, as is known in the art. The plant growth medium of the invention is stable in view of the low levels of moisture, microbial agents and degradative enzymes thereof, and microbial nutrients.

By “moisture retention” we include the ability of a material, such as the plant growth medium of the invention, to hold water after being allowed to drain. Preferably, moisture retention is measured as the retention of moisture (e.g. water) at a pressure of 0.1 bar.

By “electrical conductivity” we include the ability to conduct electricity as measured with a conductivity meter. Preferably, the electrical conductivity of the plant growth medium of the invention is between 10 mS/m and 170 mS/m; conveniently between 10 mS/m and 150 mS/m; even more preferably between 50 and 85 mS/m.

By “bulk density” we include the mass of the material at a defined moisture content divided by the volume of the same material. By “dry bulk density” we include the mass of the material present at a defined moisture content less the weight of the accompanying moisture, divided by the volume of the same material.

By “lignin content” we include the level of lignin as measured by standard chemical methods such as the Klason method (i.e. “Klason lignin”) and the DFRC method, as known in the art.

By “air filled porosity value” we include the volume of air which the material holds after free-drainage of saturating water.

Preferably, the plant growth medium of the invention comprises or consists of particles of 0.5 cm or less in length and/or diameter and/or a particles of 0.5 cm to 10 cm in length.

Methods for determining each of the above characteristics or properties of the plant growth medium are known in the art.

It will be understood that the interrelationship of each of the characteristics or properties of the plant growth medium described above, in addition to those characteristics themselves, is important in producing a plant growth medium.

Most preferably, the plant growth material generated by the method of the invention is a peat-substitute material.

In a second aspect, the invention provides a plant growth medium obtained or obtainable by the method of the invention.

In a third aspect, the invention provides a peat-substitute material comprising or consisting of a plant growth medium according to the invention.

As discussed above, the present invention may be used to produce a plant growth medium having characteristics of peat (including its physical consistency, decomposition stability, bulk density, electrical conductivity, pH, composition, lignin content and its biochemical and microbiological characteristics, among others).

Thus, by “peat-substitute material” we include a material that exhibits the same characteristic or property as (or a substantial similarity to) one or more characteristic or property of peat, thereby allowing the peat-substitute material to be successfully or effectively used instead of peat in an application in which peat is typically used or required. For example, the peat-substitute material of the invention may exhibit the same characteristic or property as (or a substantial similarity to) one or more characteristic or property of peat, including its physical consistency, decomposition stability, bulk density, electrical conductivity, pH, composition, lignin content and its biochemical and microbiological characteristics, among others.

Where the plant growth material is a peat-substitute material, it may be used instead of peat in applications that typically use, or have historically used, peat.

For example, the peat-substitute material may be used as a fuel (for example, as a bio-fuel), because it is well known that peat can be used as a solid or substantially-solid fuel that is burned to generate energy (for example, in power stations to generate electricity). Use of the peat-substitute material in that manner will therefore reduce reliance on energy-generation using non-renewable energy sources, such as peat and fossil fuels (for example, natural gas and coal). It will be appreciated that the peat-substitute material of the invention may need to be treated to remove sufficient liquid or moisture to permit its combustion before it can be used as a solid or substantially solid fuel (such as a biofuel).

In another embodiment, the peat-substitute material may be used as a product for storing and/or sequestering carbon. It is well known that peat is a carbon-containing material in which carbon has been stably stored or sequestered for many thousands of years. The peat-substitute material of the invention provides a product that is rich in carbon—furthermore, the stability of that material to microbial degradation make it suitable for storing that material (and, accordingly, the sequestered carbon) on a long-term (e.g. geo-physical) time-scale. Storage of the material could, for example, be performed in underground mines.

Thus, in an embodiment, the present invention may be used to sequester carbon obtained from bioorganic matter (such as plant matter) into a stable product that can be stored, thereby removing carbon from the carbon cycle (and potentially providing a means for reducing levels of atmospheric carbon dioxide responsible for global warming).

In a fourth aspect, the invention provides a kit for performing the method of the invention, the kit comprising one or more of the following:
a) a vessel for subjecting bioorganic matter to conditions capable of melting and/or hydrolysing and/or solubilising lignocellulose in the bioorganic matter, such as steam explosion apparatus;
b) a vessel for forming bio-alcohol from one or more sugar by fermentation, such as a bioreactor (for example, as described in WO 2008/084210);
c) bioorganic matter as described herein;
d) one or more microbial agents capable of forming bio-alcohol from one or more sugar by fermentation, as described herein; and
e) instructions for performing the method of the present invention.

The listing or discussion in this specification of an apparently prior-published document should not necessarily be taken as an acknowledgement that the document is part of the state of the art or is common general knowledge.

Preferred, non-limiting examples which embody certain aspects of the invention will now be described:

### EXAMPLES

### Example 1

### Experimental Data

Production and evaluation of a growing medium from the recalcitrant material produced by partial saccharification of steam-exploded wheat straw.

### Materials and Methods

Wheat straw (chopped to approximately 10 cm lengths) was obtained from Dixon Brothers, Rickinghall, Norfolk, IP22 1LY.

### Pre-Treatment and Saccharification

### Steam Explosion:

1 kg batches of chopped wheat straw were steam-treated for 10 minutes at 180° C., 190° C., 195° C., 200° C., 210° C. and 230° C. and exploded into 6.7 L of water at 50° C.
The straw and liquor were separated on 0.1 mm nylon mesh using a spin-drier.
The steam-exploded straw was kept wet and frozen until required.

### Saccharification:

Thawed steam-exploded straw was digested in 4 L batches at 5% substrate concentration using a cocktail of cellulases and xylanases (N11/7 & N11/9; Biocatalysts Ltd.) in 50 mM NaOAc pH 5 at 50° C. and 180 rpm for 42 h. The cocktail comprised 2.5% for N11/7 and 1.25% for N11/9, (with respect to dry matter of substrate).
The mixture was filtered through 1.32 mm nylon mesh and dewatered in a domestic spin drier. The recalcitrant retentate collected on the 1.32 mm mesh was labelled ‘coarse’.
The filtrate was re-filtered through 0.1 mm mesh (the retained material was labelled “fines”). The final filtrate was centrifuged at 4200 rpm for 10 min. and labelled “<0.1 mm”.
Solid fractions and the hydrolysate were stored wet at −40° C. The liquor from the steam-explosion was also retained frozen.
The effect of time and temperature are combined as a severity factor (Overend & Chornet 1987):

Severity Factor=Log₁₀(time×exp((Temperature−100)/14.75))

### Analyses:

The dry mass of each solid fraction was determined by weighing duplicate samples of wet material at 40° C. for 16 h.
The glucose monosaccharide content of the hydrolysate was measured using a colorimetric assay (GOPOD Megazyme).

### FTIR Spectroscopy

All samples for FTIR spectroscopy were freeze-milled for 3 min to pulverise them. Spectra were acquired in triplicate with 64 scans from 800 to 4000 cm⁻¹. The spectra were truncated to 800 to 1800 cm⁻¹, base-line anchored to 1800 cm⁻¹ and normalised.

### Physical Testing for Growing Media Criteria

Tests for dry matter, wet bulk density, air-filled porosity and moisture retention at 0.1 bar pH and conductivity are as described in WO 2008/084210. Dry bulk density is calculated from the wet bulk density, moisture content, and dry matter, ideally from welt bulk density and dry matter content.

### Evaluation of Growing Media for Plant Propagation

Coarse Material:
For each coarse sample, material which had been used for the measurement of air-filled porosity was combined with any remaining which had not been used for physical testing. The combined sample was soaked in excess water for 6 h and spin-dried in a 1.32 mm nylon mesh. The compacted pellets were dispersed by hand. The dry matter content was determined by drying small samples at 40° C.
Fines:
The fines were wetted and formed into blocks.
   Germination Trials with Lettuce (Lactuca sativa) Seeds
Testing Coarse Material:
Preliminary growing trials were carried out using lettuce seeds. The recalcitrant coarse material from wheat straw steam-exploded at 210° C. (18.1 bar) for 10 min and digested with Biocatalysts enzymes for 42 h was tested directly and in a 50:50 blend with a commercial compost. Growing material was placed in 7 cm pots. Seeds (Lactuca sativa cv “Rosetta” or cv “Unrivalled” [Suttons Seeds, UK]) were planted at a depth of 1 cm. Two seeds were placed in each pot. Each treatment consisted of four pots (i.e. 8 seeds per treatment). The pots were placed in a growth room with 24 h light. The time of emergence was noted.
   Germination Trials with Cucumis sativus Seeds
Preparation of Growing Media for Germination Trials:
Trials were carried out on moistened substrates as follows: Substrates were tested as provided or after further washing or milling or both. Washing was carried out by immersing the substrate (1 litre) in water (2 litres), standing for 30 mins, and then allowing to drain until all free water had been removed. This was carried out twice. Milling: 300 g of sample was milled to less than 3 mm (assessed by sieving) using a homogenizer (Moulinex—Chopper La Moulinette, 800 watts). This resulted in a relatively uniform structure in comparison with the range of structures (greater than 1.32 mm) provided.
Growing Conditions:
Transparent, rectangular plastic boxes (122×82×82 mm; Hofstatter & Ebbesen A/S, model 500/80, Espergarde, Denmark), were each filled with 200 ml growing media which was conditioned with distilled water. Cucumber (Cucumis sativus) seeds (15 per box replicate) were sown directly on top of the material. The boxes were placed in a germination cabinet (Friocell 111, MMM Medcenter Einrichtungen GmbH, München, Germany). Two replicate boxes were evaluated for each treatment. Germination was assessed over a 2 day period at 20° C. (dark)/30° C. (light) and the relative humidity within the boxes was maintained close to 100%.
Evaluation of Germination and Seedling Development:
A seed was considered to have germinated when the radicle protruded about 2 mm. After the germination test, the boxes were maintain at same conditions until the cotyledons had opened. After 7 days of incubation the phytotoxicity impact of the materials (treatments) were evaluated and the seedling performance (top, root and colour) was quantified by rating each plant on a scale of zero to five as follows:
0—Seedling dead;
1—Seedlings showed symptoms of stress;
2—Seedling that showed extremely little growth since germination;
3—Slow growth;
4—Healthy seedling exhibiting a large amount of growth;
5—Exceptional growth and fullness.

The quantified seedling performance (based on 20 seedlings) was described as follows:

| Quantified seedling performance | |
|---|---|
| rating (base on 20 seedling) | Seedling growth category |
| 5.0-4.0 | High |
| <4.0-3.25 | Medium |
| <3.25 | Low |

### Results

### Effect of Steam Explosion on Saccharification and Breakdown of Wheat Straw

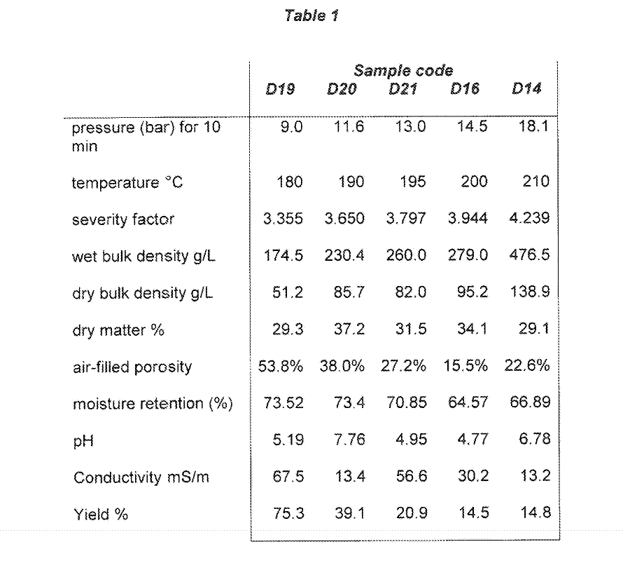

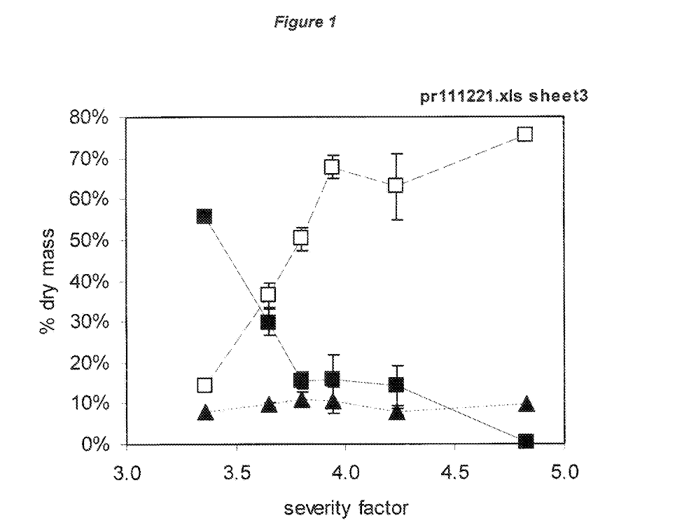

FIG. 1 shows the impact of severity factor on the enzymatic release of glucose, and the levels of differently-sized residues as a % dry mass in the digestates.

The proportion of coarse recalcitrant material (square black points) declined with severity of pre-treatment, being negligible at 230° C. Hence further studies focused on material produced at severities less than 4.5.

As a control, 4 samples of wheat straw steam-exploded at 180° C., 190° C., 195° C. and 210° C. were incubated at 5% substrate concentration in 50 mM NaOAc pH 5 at 50° C. and 180 rpm for 42 h without enzymes. The mass fraction of coarse material was 98% for straw steam-exploded at 180° C., 190° C. and 195° C. and 95% for straw exploded at 210° C. and saccharification was negligible in all 4 samples. Hence, the effect of steam explosion on the production of degraded coarse and fine material is manifest through the enzymatic digestion. Steam explosion on its own has little impact on the level of coarse material.

Up to 30% of the constituent cellulose was digested to glucose, but glucose concentrations were low, generally less than 33.8 mM. This may be too low for fermentation on its own. However, it may be useful for addition to the water stream in first generation plants. However, the potential for increasing glucose concentration through further saccharification of the fines is shown below.

### Chemical Analysis

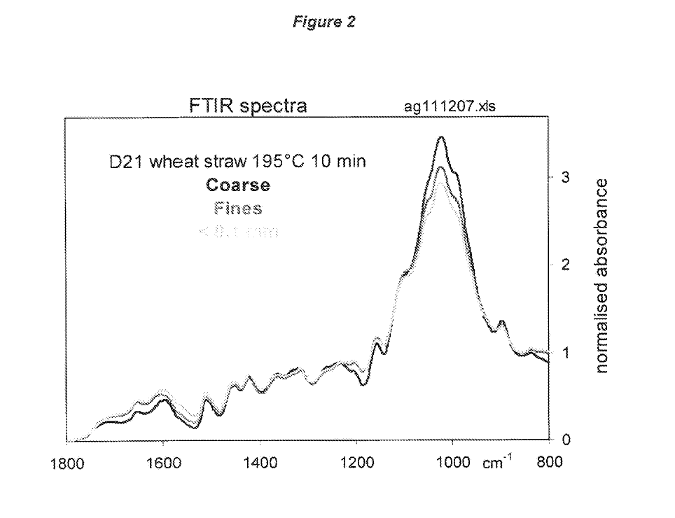

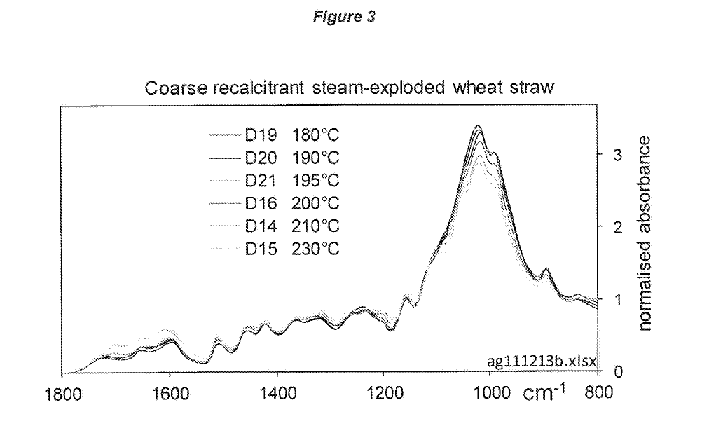

The FTIR spectra have been acquired of all the coarse and fine materials from the straw steam-exploded at 195° C., as shown in FIGS. 2-4. FIG. 2 compares FTIR spectra of coarse and fine material from pre-treated (195° C., 10 min) and digested wheat straw. The decrease in the overall intensity of absorbance around 1000 cm⁻¹ indicates that the fines contain relatively less carbohydrate than the coarse material. It is likely that the fines result from the loss of predominantly hemicellulosic materials which are involved in cell adhesion. Their degradation results in enhanced cell separation, thus leading to the production of fine particles. FIG. 3 compares the coarse material produced after digestion of straw pre-treated across the range 180-230° C. This also shows that at higher levels of pre-treatment, the polysaccharide complement in the coarse material is reduced. This is likely to be due to the enhanced loss of arabinoxylan hemicelluloses at the higher temperatures. They are likely to be present in hydrolysed form in the steam explosion liquor and in the enzyme hydrolysis liquor.

### Effect of Steam Explosion and Digestion on Physical Properties of Coarse Material

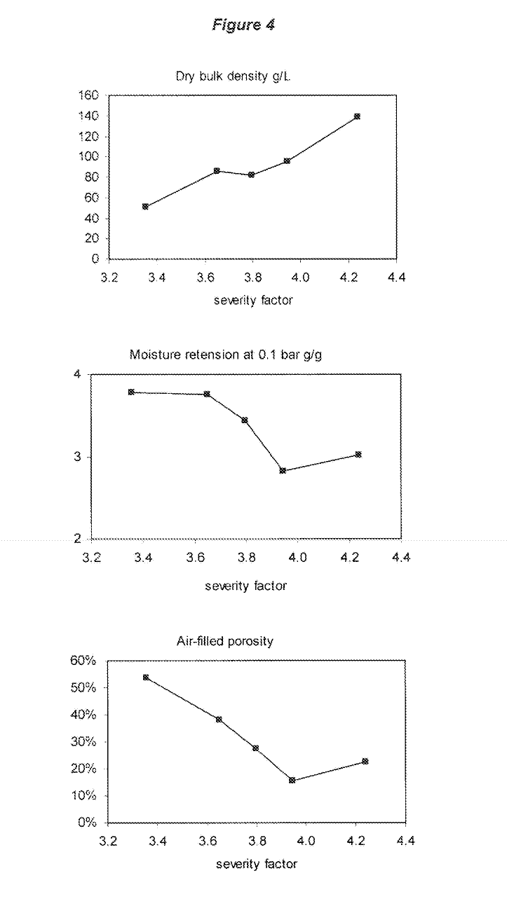

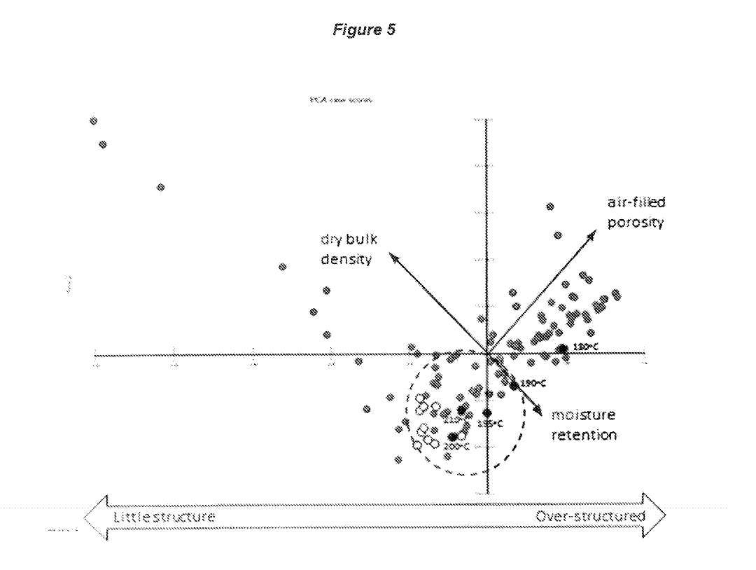

The coarse materials produced as described above from wheat straw that had been pre-treated at 180-210° C. were evaluated for physical properties that relate to the quality of growing media substrates. In particular, dry bulk density, air-filled porosity and moisture retention have been previously identified (ref patent WO 2008/084210) as key to growing media structural quality characteristics. In addition, pH, conductivity, wet bulk density and dry matter were also measured. The results are shown in Table 1 and FIG. 4.

Table 1 presents the physical properties of “coarse” material produced after enzymatic digestion of pre-treated (by steam explosion) wheat straw, as a function of pre-treatment temperature. The separate samples are referred to as D19, D20, D21, D16 and D14.

FIG. 4 shows that as pre-treatment temperature increases, the dry bulk density of the coarse material is increased, and this is accompanied by a decrease in air-filled porosity and moisture retention. This is due to the reduction in structural integrity and a reduction in particle size (albeit still greater than the 1.32 mm sieve size). The dry bulk density is of particular importance because it reflects the amount of dry matter present in a given volume of moist coarse material. This is analogous to the changes that occur during the microbial/enzymatic degradation of plant material during the composting process. We have previously shown that the changes in plant structure during composting can be related to growing material quality, and compared with high quality growing media such as peat-based media, using principal components analysis of the three parameters shown in FIG. 4. Such an analysis for the coarse materials under study is shown in FIG. 5 in relation to many other growing media.

The three structure-related measurements (dry bulk density, moisture retention and air-filled porosity) have been evaluated by Principal Components Analysis (PCA) and are shown in FIG. 5 compared with many other materials (grey-filled circles). Points (samples of plant material/growing media) on the far right hand side are very highly structured, reflecting the raw plant structure of poorly- or un-degraded plant material. Points on the far left hand side of the PCA graph are materials that are highly degraded and soil like. These materials have been degraded by composting to bacterial and fungal biomass. They exhibit virtually no structure and hence have very high dry bulk densities (due to close packing of the particles) and poor moisture retention and air-filled porosity. Between these two extremes lie plant materials (composts and growing media) that have been degraded to varying extents. Of particular note is the group of materials within the bounds of the dotted circle. This area contains materials the structures of which are most similar to the highest quality growing media including peat-based growing media (shown as open circular points). He black circles show the positions of the coarse materials described above. They show that as the severity of pre-treatment increases, the coarse material becomes more degraded, moving from the right side of the PCA graph down into the dotted circle of potentially high quality material, close to peat-based growing media.

The results therefore show that at lower temperature, (180° C. and 190° C.) the material is highly structured. However at above 195° C., the coarse material is in the area that is particularly suitable for producing useful growing media.

### Evaluation of Growing Media Potential of Recalcitrant Coarse Material.

Germination Studies Using Lettuce (Lactuca sativa) Seeds:

Two small-scale growing trials were performed with lettuce seeds on coarse recalcitrant material from straw steam-exploded at 210° C.

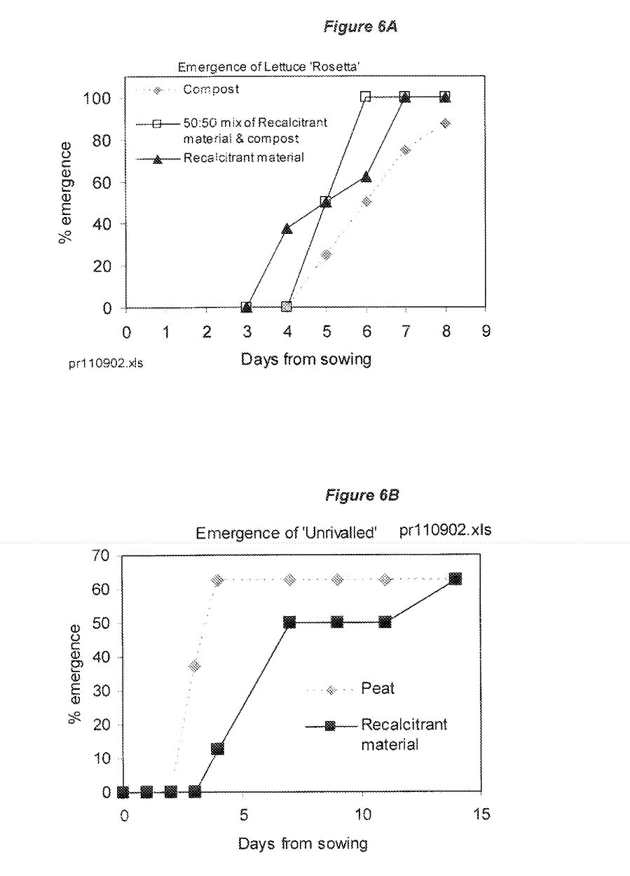

Lettuce Test 1: Results for lettuce cv “Rosetta” are shown in FIG. 6A. 100% emergence (8 plants) was achieved for both the coarse material on its own, and also in a 50:50 mix with a commercial compost-based growing medium.

Lettuce Test 2: Results for lettuce cv “Unrivalled” grown in an environmental chamber (Sanyo MLR-350) at 23° C. with continuous light. The results are shown in FIG. 6B and show that in comparison with peat, 100% germination occurs slightly later.

### Germination Studies Using Cucumber Seeds:

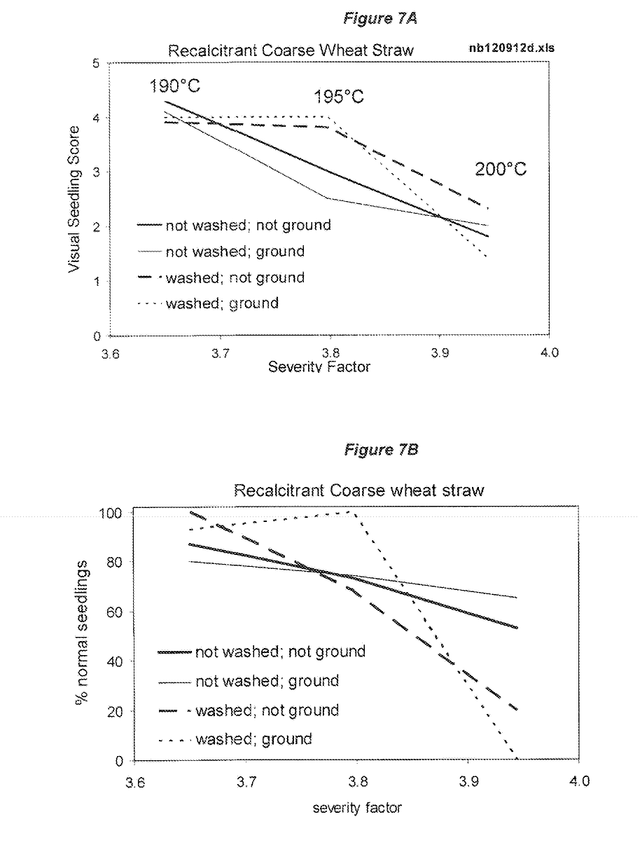

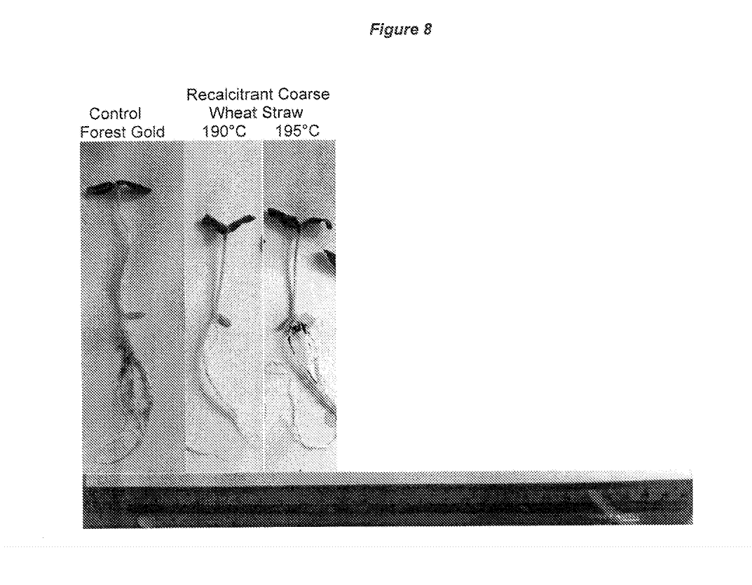

Coarse material from recalcitrant residues of wheat straw that had been pre-treated at 190° C., 195° C. and 200° C. were evaluated for phytotoxicity using a germination test with cucumber seedlings. The results showed that for raw coarse materials at 190° C. and 195° C., seedlings showed 100% germination; at 200° C. this dropped to 80%. This was accompanied by a decrease in the visual seedling score (FIG. 7A) and the % seedlings showing normal form. Hence, higher severities result in coarse material which exhibits some degree of phytotoxicity. However, this is not of significance in coarse material produced from digests of material pre-treated at 190° C. or 195° C. Further washing of the material improves the score somewhat indicating that this might be a route to improve the media. It is possible that the phytotoxicity results from breakdown products produced during thermophysical pre-treatments such a furfurals. These are known to have antimicrobial activity and are more prominent after higher temperature pre-treatments. It is possible that they effect seed germination.

### Example 2

### Experimental Data

Evaluation of the potential to saccharify fines produced from the saccharification of pre-treated wheat straw.

### Materials and Methods

### Pre-Treatment:

Wheat straw (as for Experiment 1) was pre-treated at 195° C. for 10 min.

### Enzymatic Digestion:

### Primary Digestion to Produce Fines:

The pre-treated wheat straw was then digested at a substrate concentration of up to 15% (w/v) using Novozymes Cellic HTec2 for 24 h in a stirred bioreactor in 50 mM NaOAc pH 5 at 50° C. The fines were recovered as for Experiment 1.

### Secondary Digestion to Saccharify Fines:

Fines were re-digested at a range of substrate and enzyme concentrations with Cellic CTec2 and a 10% substitution with Cellic HTec2 (Novozymes) in 50 mM NaOAc pH 5 at 50° C. Digestions were performed in 50 mL plastic pots each containing a 1 inch ceramic or steel ball, on their side in an incubator at 90 rpm.

### FTIR Evaluation

As for Experiment 1.

### Sugars Analysis

Gas chromatography of alditol acetates produced after hydrolysis in acid using standard methods (see previous patent).

### Results:

### Provision of Coarse and Fine Fibre

Creation of fines: using a high shear bioreactor, pre-treated material was readily degraded using Cellic HTec2 to produce coarse and fine materials which were separated on a 1.32 mm sieve as in Expt1. The ratio of coarse to fine material was affected by time, stirring and enzyme concentration. Cellic HTec2 is a cocktail of hemicellulose enzymes, and contains little cellulase. Hence, production of coarse and fine material did not result in the release of much glucose in contrast to Experiment 1.

The chemical compositions of the coarse and fine materials were similar in that they both contained glucose (cellulose) at about 350 ug/mg, and xylose at about 50 ug/mg. The FTIR spectra were also similar.

### Saccharification of Fines Using Multiple Additions:

Because fines have little fibrous structure, they may be stirred effectively at much higher concentrations than coarse fibre. This study assessed the saccharification of fines at an initial substrate concentration of 15% (w/v) with Cellic CTec2 and Cellic HTec2 at a total of 1.5%, 3%, 10% and 30% (w/w with respect to substrate).

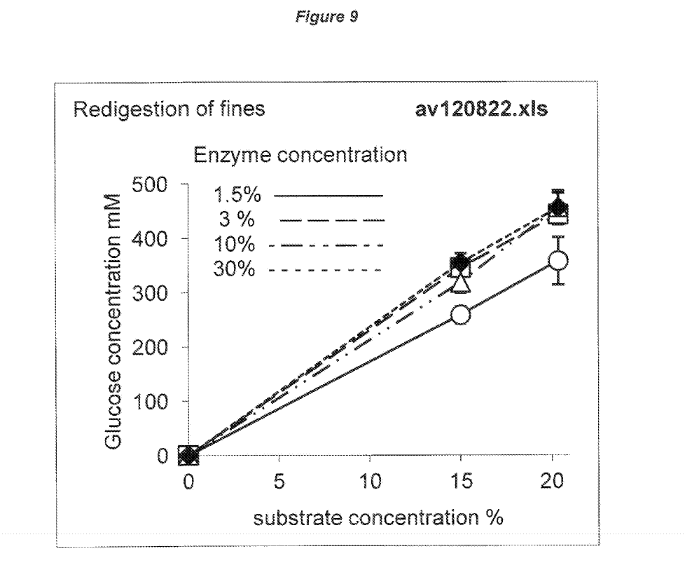

The first digestion was carried out for 3 days at which point the same amount of enzyme and substrate was added again and digested for another 3 days. The results are shown in FIG. 9.

The results show that saccharification of fines at a high substrate concentration (which is increased by addition) using the Novozymes enzymes permits a high level of glucose to be realised. The approach facilitates a substrate concentration of over 20% (w/v) and a glucose concentration of nearly 500 mM (86 to 89% recovery) for enzyme concentration of 3% or greater. 0.5M glucose equates to about 9% (w/v) which will give rise to approximately 4.5% (v/v) ethanol after fermentation with yeast.

### Conclusions:

Lignocellulosic material can be pre-treated using e.g. steam explosion, and then partially-digested using cell wall degrading enzymes to produce glucose for bio-ethanol production and structured material suitable for use as a growing medium or growing medium supplement. Fine particles produced during the process may be further saccharified to at high substrate concentration to produce glucose to concentrations of over 0.5 mol/l. The ratios of the glucose and growing media outputs may be modulated by changing the pre-treatment and enzymatic digestion conditions. The quality of the final growing media can be controlled by controlling the above processing conditions, and monitoring three key physical parameters—air-filled porosity, dry bulk density, and moisture retention.

An outline of the preferred steps of the method of the invention is shown in FIG. 16.

### Example 3

### Experimental Data

Development of process for producing coarse and fine materials using commercial cell wall degrading enzymes to facilitate scale up of growing media preparation from steam-exploded wheat straw using a bespoke bioreactor vessel.

The aim of this series of experiments was to:
(a) Develop methods to utilise enzymes that would enable the fragmentation of steam exploded wheat straw into coarse and fines whilst minimising the release of glucose;
(b) Develop scale up procedures to produce Kg quantities and 100 Kg quantities of coarse material for further plant growth trials.

### Materials and Methods

### Pre-Treatment:

1 kg batches of chopped wheat straw were steam-treated for 10 minutes at 190° C., and exploded into 6.7 L of water at 50° C.
The straw and liquor were separated on 0.1 mm nylon mesh using a spin-drier.
The steam-exploded straw was kept wet and frozen until required.

### Saccharification:

The pre-treated wheat straw was digested at a substrate concentration of 13% (w/v) using Novozymes Cellic HTec2 hemicellulase (Novozymes) for 24 h in a stirred bioreactor (Elliston et al., 2013, Bioresource Technology, 134:117-126) in 50 mM NaOAc pH 5 at 50° C.
The coarse and fines materials were recovered as for Experiment 1.

### Other Analyses:

All analyses are as described in Experiment 1.

### Results

### Effect of Enzyme Loading on Coarse and Fines Production

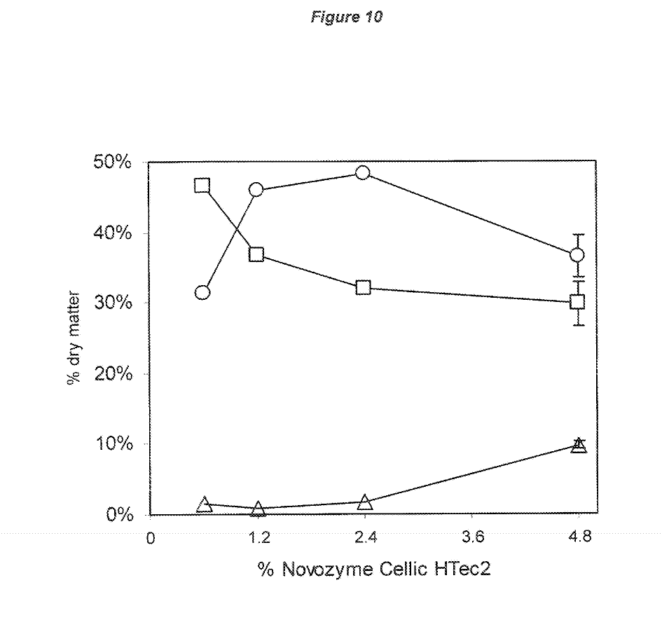

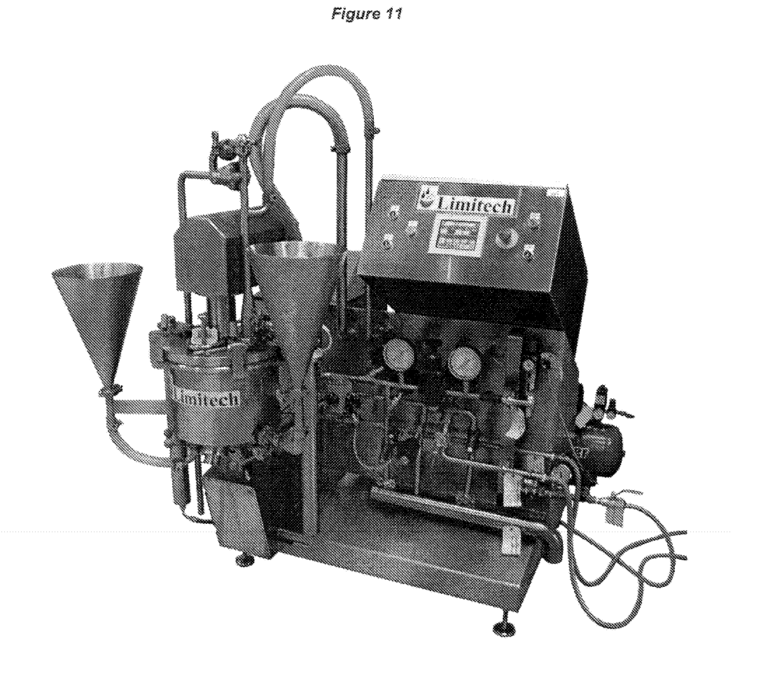

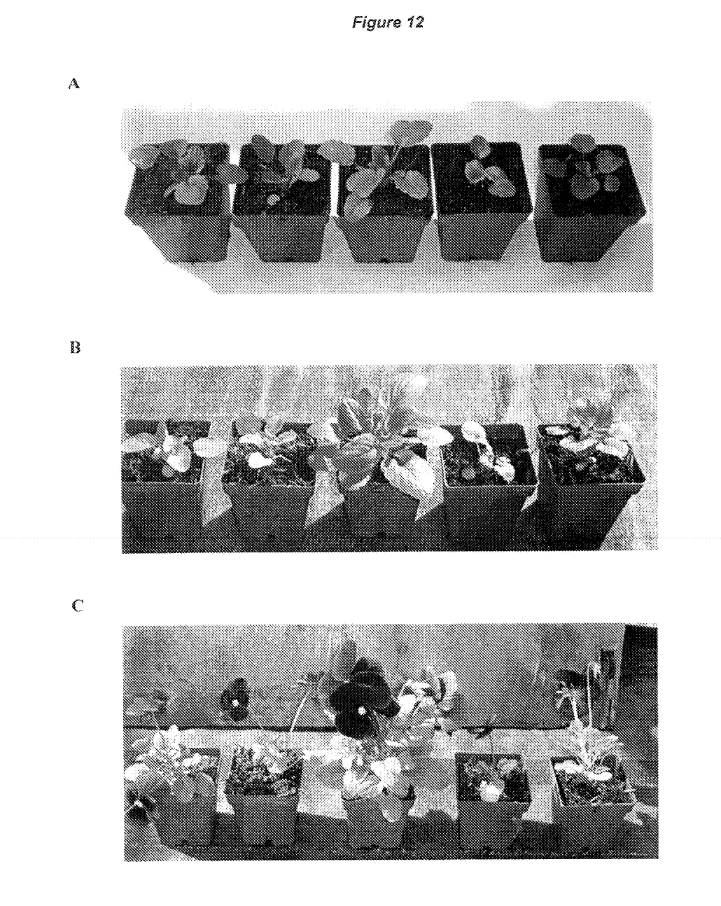

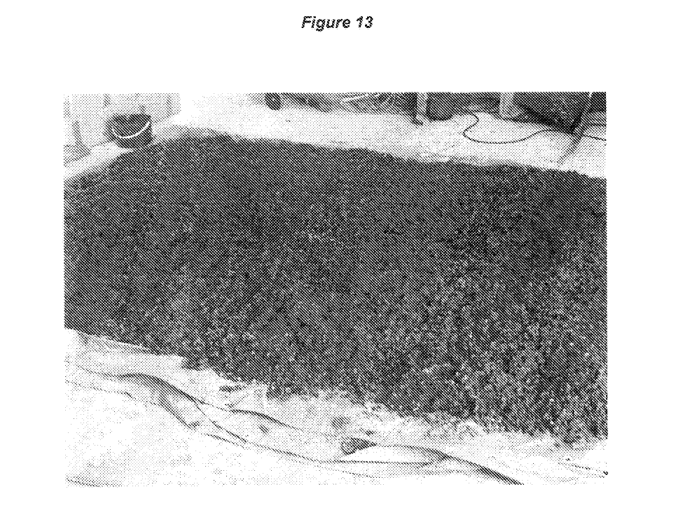

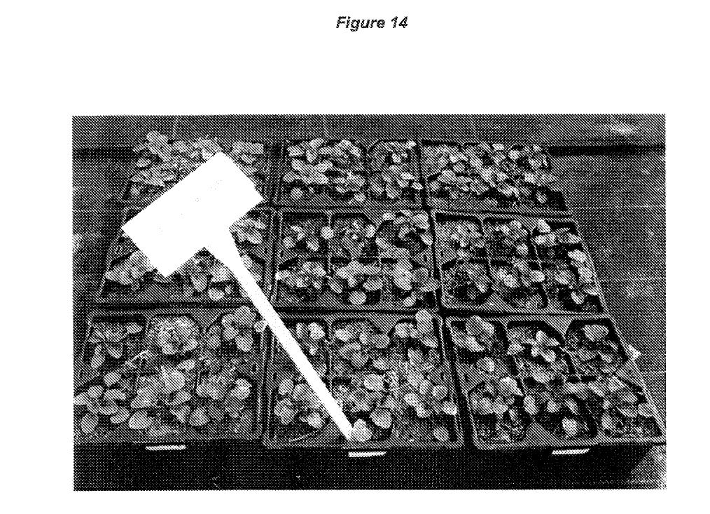

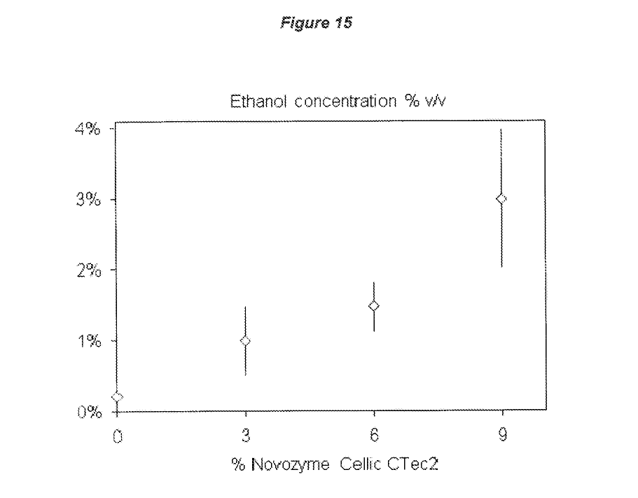

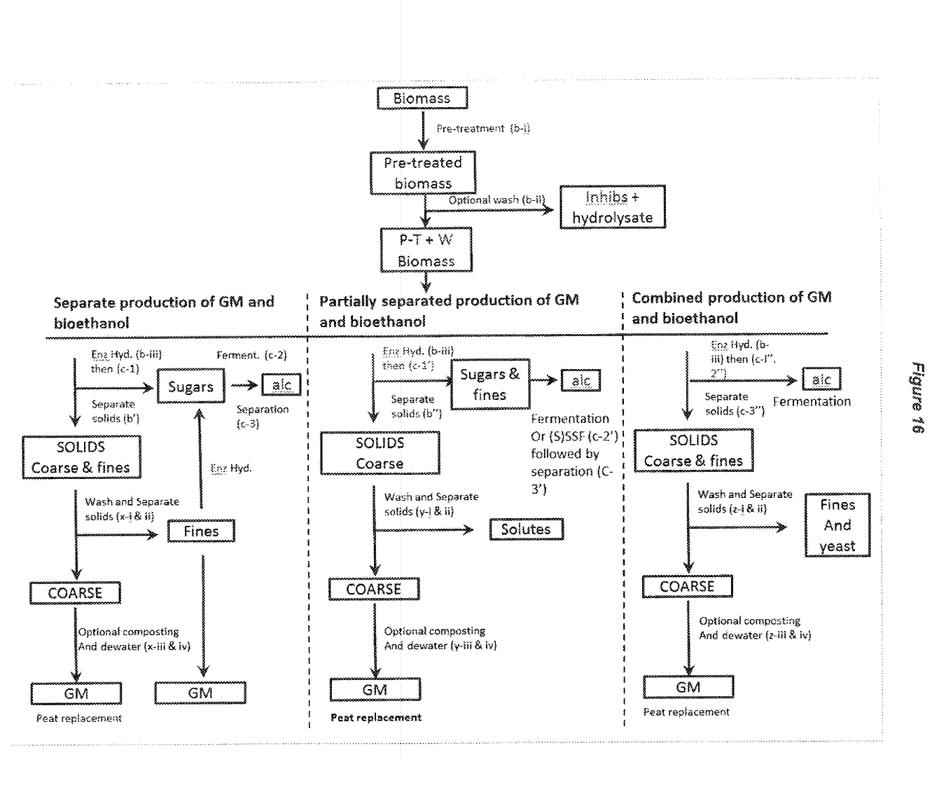

FIG. 10 shows the dry matter proportions of coarse and fines materials and glucose obtained from Cellic HTec2 digestions of wheat straw steam exploded at 190° C. for 10 min. This was carried out in a bioreactor (as shown in FIG. 11; a suitable bioreactor is also discussed in WO 2008/084210 and Elliston et al., 2013, Bioresource Technology, 134:117-126) which enabled a substrate concentration of over 20% (w/v) to be handled.

The bioreactor used (and shown in FIG. 11) comprises: a heating means that is capable of heating and maintaining the reactants at a temperature suitable for correct functioning of the enzymes (for example, 30-70° C., and preferably 50° C.); and means for mixing the reactants at between 10-65 RPM, the rate of which is dependent on the stiffness of the reactants being mixed.

The bioreactor was run using the following conditions: a temperature of 50° C. and mixing at 39 RPM, for 24 hours. The reaction included 50 mM acetate as a buffer at pH 5.

An enzyme loading of 1.2% (wt/wt cellulose) was chosen for further scale up work. The results indicated that an enzyme loading of 1.2% (wt/wt cellulose) was suitable for production of coarse and fines with minimal glucose release.

### Small Pilot Scale Bioreactor Production of Coarse Material

Several bioreactor runs were performed using the chosen enzyme loading. Physical properties were measured of coarse recalcitrant material obtained using 1.2% Cellic HTec2: wet bulk density, 245 g/L; dry bulk density 72 g/L; moisture content 70%.
As a result, the following samples were sent to Bulrush Horticulture for assessment using viola plants (FIG. 12):
5 L of wheat straw steam-exploded at 11.5 bar 190° C. for 10 min. (0.96 kg wet mass, moisture content 59%, wet bulk density 137 g/L, dry bulk density of 56 g/L)
5 L prepared in the bioreactor of recalcitrant coarse residue from a 1.2% Cellic HTec2 treatment of wheat straw steam-exploded at 11.5 bar 190° C. for 10 min (1.58 kg wet mass, moisture content 68%, wet bulk density 187 g/L, dry bulk density of 60 g/L)

### Plant Growth Trials

FIG. 12. Shows viola plants at approximately 3, 5 and 7 weeks grown in un-treated coarse materials in comparison with standard multipurpose growing media.
All plants were healthy. The coarse-material was clearly suitable for promoting and maintaining plant growth. The coarse-material-grown plants were not quite as advanced as those grown in the multipurpose compost, which is likely to be due to the lack of additional nutrients used in commercial mixes.

Scale-up of growing media production was then carried out (Example 4).

### Example 4

### Experimental Data

Large scale production and evaluation of growing medium from the recalcitrant material produced by partial saccharification of steam-exploded wheat straw.

### Materials and Methods.

### Pre-Treatment:

100×1 kg (Dry Weight) batches of chopped wheat straw were steam-treated for 10 minutes at 190° C., and exploded into 5 L of water at 50° C.
The straw and liquor were separated on 0.1 mm nylon mesh using a spin-drier.
The steam-exploded straw was kept wet and frozen until required.

### Saccharification:

The pre-treated wheat straw was digested at a substrate concentration of 10% (w/v) using Novozymes Cellic HTec2 hemicellulase (Novozymes) for 24 h in a large high-torque bioreactor in 50 mM NaOAc pH 5 at 50° C. for 24 h.
The coarse and fines materials were recovered as for Experiment 1.

### Results

The large scale production of coarse material is shown in FIG. 13

The following was sent to Bulrush Horticulture for trials:
219 L prepared in a bioreactor of recalcitrant coarse residue from a 1.2% Cellic HTec2 treatment of wheat straw steam-exploded at 11.5 bar 190° C. for 10 min (75.716 kg wet mass, moisture content 72%, wet bulk density 345 g/L)

Initial trials (FIG. 14) indicated that the material was capable of supporting seed germination and plant growth, to a similar level as in Example 3.

### Example 5

### Experimental Data

### Production of Bioethanol from Fines

### Materials and Methods

Fines were obtained from pre-treated wheat straw. This involved 1.2% HTec2 treatment of wheat straw that had been steam-exploded at 11.5 bar 190° C. for 10 min.

Enzymatic digestion and fermentation (SSF) of fines with CTec2 at a range of concentrations and yeast was carried out at 30% (w/v) substrate concentration over 6 days. The incubation involved continuous shaking in an incubator at 40° C. and 200 rpm. Continuous agitation was achieved by inclusion of a 1 inch steel ball in each plastic pot.

### Results

The effect of enzyme concentration on the yield of ethanol through SSF is shown in FIG. 16. From the cellulose content the maximum possible EtOH is 5.9%.

51% conversion to ethanol was obtained on average at the highest enzyme concentration.

### Conclusions from Examples 1-5

Lignocellulosic material can be pre-treated using e.g. steam explosion, and then partially-digested using cell wall degrading enzymes to produce glucose for bio-ethanol production and structured material suitable for use as a growing medium or growing medium supplement. Fine particles produced during the process may be further saccharified to at high substrate concentration to produce glucose to concentrations of over 0.5 mol/l. The ratios of the glucose and growing media outputs may be modulated by changing the pre-treatment and enzymatic digestion conditions.

The quality of the final growing media can be controlled by controlling the above processing conditions, and monitoring three key physical parameters—air-filled porosity, dry bulk density, and moisture retention.

An outline of the preferred steps of the method of the invention is shown in FIG. 16.

1. A method of producing one or more sugar for bio-alcohol production, comprising the step of degrading bioorganic matter comprising lignocellulose, to generate one or more sugar from the lignocellulose and a degraded bioorganic residue; characterised in that the method further comprises the step of forming a plant growth medium from the degraded bioorganic residue.

2. A method according to claim 1 further comprising the step of producing bio-alcohol from the one or more sugar.

3. A method according to claim 2 comprising the following steps:
(a) providing an amount of bioorganic matter comprising lignocellulose;
(b) degrading the bioorganic matter to generate one or more sugar from the lignocellulose and a degraded bioorganic residue; and
(c) forming bio-alcohol from the one or more sugar;
wherein the method further comprises the step, performed after step (b) or after step (c), of forming a plant growth medium from the degraded bioorganic residue.

4. A method according to claim 1 wherein the degraded bioorganic residue comprises a structure capable of supporting plant growth.

5. A method according to claim 1 wherein degrading the bioorganic matter comprises the step of:
(b-i) subjecting the bioorganic matter to conditions capable of melting and/or hydrolysing and/or solubilising some or all of the lignocellulose in the bioorganic matter.

6. A method according to claim 5 wherein the conditions in step (b-i) comprise heating at a temperature of between approximately 100° C. and approximately 240° C., preferably at a temperature of between approximately 190° C. and approximately 240° C.

7. A method according to claim 6 wherein the conditions in step (b-i) comprise heating at a temperature of: approximately 100° C.; or approximately 110° C.; or approximately 120° C.; or approximately 130° C.; or approximately 140° C.; or approximately 150° C.; or approximately 160° C.; or approximately 170° C.; or approximately 190° C.; or approximately 190° C.; or approximately 200° C.; or approximately 210° C.; or approximately 220° C.; or approximately 230° C.; or approximately 240° C.

8. A method according to claim 5 wherein step (b-i) is performed using steam explosion.

9. A method according to claim 5 wherein step (b-i) is performed by a method selected from the group consisting of: hot-water treatment; AFEX (Ammonia Fibre Explosion or Ammonia Fibre Expansion); extrusion; and autoclaving.

10. A method according to claim 5 wherein degrading the bioorganic matter further comprises the steps, performed after step (b-i), of:
(b-ii) optionally, washing the treated bioorganic matter;
(b-iii) subjecting the treated bioorganic matter to conditions capable of degrading plant cell walls in the bioorganic matter.

11. A method according to claim 10 wherein the conditions in step (b-iii) comprise contacting the bioorganic matter with one or more enzyme selected from the group consisting of: a cellulase; a hemicellulase; a pectinase; an esterase; a protease; a xylanase; and an oxido-hydrolase.

12. A method according to claim 3 wherein the one or more sugar generated from the lignocellulose is glucose and/or cellobiose and/or xylose and/or arabinose and/or mannose and/or galactose and/or glucuronic acid and/or galacturonic acid and/or fucose and/or rhamnose.

13. A method according to claim 3 further comprising the step, performed after step (b), but before step (c), of:
(b′) separating the degraded bioorganic residue from the one or more sugar generated from the lignocellulose.

14. A method according to claim 13 wherein forming a plant growth medium from the degraded bioorganic residue comprises the steps, performed after step (b′), but before step (c), of:
(x-i) providing the degraded bioorganic residue, generated by step (b′);
(x-ii) washing the degraded bioorganic residue;
(x-iii) optionally, subjecting the degraded bioorganic residue to conditions capable of decomposing the bioorganic residue, and inhibiting decomposition prior to its completion;
(x-iv) removing moisture from the resulting degraded bioorganic residue.

15. A method according to claim 13 wherein step (c) comprises the steps of:
(c-1) providing the one or more sugar generated from lignocellulose, generated by step (b′);
(c-2) forming bio-alcohol from the one or more sugar by fermentation;
(c-3) optionally, separating the bio-alcohol from the fermentate.

16. A method according to claim 15 wherein fermentation is performed by contacting the one or more sugar with one or more microbial agent.

17. A method according to claim 3 further comprising the step, performed after step (b), but before step (c), of:
(b″) separating the “coarse” fraction of the degraded bioorganic residue from the “fine” fraction of the degraded bioorganic residues and the one or more sugar generated from the lignocellulose.

18. A method according to claim 17 wherein forming a plant growth medium from the degraded bioorganic residue comprises the steps, performed after step (b″), but before step (c), of:
(y-i) providing the “coarse” fraction of the degraded bioorganic residue, generated by step (b″);
(y-ii) washing the “coarse” fraction of the degraded bioorganic residue;
(y-iii) optionally, subjecting the “coarse” fraction of the degraded bioorganic residue to conditions capable of decomposing the bioorganic residue, and inhibiting decomposition prior to its completion;
(y-iv) removing moisture from the resulting degraded bioorganic residue.

19. A method according to claim 17 wherein step (c) comprises the steps of:
(c-1′) providing the “fine” fraction of the degraded bioorganic residues and the one or more sugar generated from the lignocellulose, generated by step (b″);
(c-2′) forming bio-alcohol from the “fine” fraction of the degraded bioorganic residues and the one or more sugar by fermentation, preferably by simultaneous saccharification and fermentation (SSF) or semi-simultaneous saccharification and fermentation (SSSF);
(c-3′) optionally, separating the bio-alcohol from the fermentate.

20. A method according to claim 19 wherein fermentation is performed by contacting the one or more sugar with one or more microbial agent.

21. A method according to claim 12 wherein step (c) comprises the steps of:
(c-1″) providing the degraded bioorganic residue and the one or more sugar from the lignocellulose;
(c-2″) forming bio-alcohol from the degraded bioorganic residue and the one or more sugar from the lignocellulose, by fermentation; and
(c-3″) optionally, separating the bio-alcohol from the degraded bioorganic residue.

22. A method according to claim 21 wherein fermentation is performed by contacting the one or more sugar with one or more microbial agent.

23. A method according to claim 22 wherein the bio-alcohol is separated from the degraded bioorganic residue by a method selected from the group consisting of: filtration (such as vacuum filtration); distillation; reverse osmosis; and partitioning the bio-alcohol to the organic phase.

24. A method according to claim 23 wherein forming a plant growth medium from the degraded bioorganic residue comprises the steps, performed after step (c-3″), of:
(z-i) providing the degraded bioorganic residue, generated by step (c-3″);
(z-ii) washing the degraded bioorganic residue;
(z-iii) optionally, subjecting the degraded bioorganic residue to conditions capable of decomposing the bioorganic residue, and inhibiting decomposition prior to its completion;
(z-iv) removing moisture from the resulting degraded bioorganic residue.

25. The method according to claim 14 further comprising the step of adding slow-release fertiliser to the plant growth medium produced in step (x-iv), preferably a slow-release fertiliser comprising or consisting of potassium and/or nitrogen and/or phosphorus.

26. The method according to claim 1 further comprising packaging the plant growth medium.

27. The method according to claim 1 wherein the bioorganic matter comprising lignocellulose comprises plant matter comprising lignocellulose, preferably selected from the group consisting of lignified plant matter and semi-lignified plant matter.

28. The method according to claim 27 wherein the lignified plant matter and/or semi-lignified plant matter comprises or consists of sheets and/or fibres of lignified plant matter.

29. The method according to claim 27 wherein the plant matter comprising lignocellulose is selected from the group consisting of: wood; wood chippings; straw;
straw leaves; cereal leaves; brewer's grain; wheat bran; oat grain; rice bran; and grasses (such as Miscanthus species).

30. The method according to claim 1 wherein the amount of bioorganic matter comprising lignocellulose is at least 10 kg, for example at least 20 kg, 30 kg, 40 kg, 50 kg, 60 kg, 70 kg, 80 kg, 90 kg, 100 kg, 150 kg, 200 kg, 250 kg, 300 kg, 400 kg, 500 kg, 10 tonnes, 20 tonnes, 50 tonnes, 100 tonnes, 200 tonnes, 300 tonnes, 500 tonnes, 1,000 tonnes, 2,000 tonnes, 5,000 tonnes, 10,000 tonnes, 20,000 tonnes, 50,000 tonnes, 100,000 tonnes, 200,000 tonnes, 300,000 tonnes, 400,000 tonnes, 500,000 tonnes, 600,000 tonnes, 700,000 tonnes, 800,000 tonnes, 900,000 tonnes, 1,000,000 tonnes or more.

31. The method according to claim 1 further comprising the step of analysing a sample of the degraded bioorganic residue to determine its physical and/or structural characteristics.

32. The method according to claim 1 further comprising the step of analysing a sample of the plant growth medium, to determine its physical and/or structural characteristics.

33. The method according to claim 1 wherein the plant growth medium exhibits one or more of the following properties:
i) no detectable decomposition or minimal detectable decomposition;
ii) a moisture retention of 55% or more at 0.1 bar; for example, 60% or 70% or 80% or 90% or more;
iii) pH 6.5 or less; for example, pH6, pH5, pH4, pH3, pH2, pH1 or less;
iv) an electrical conductivity of 422 mS/m or less; for example, 400 mS/m, 300 mS/m, 200 mS/m, 100 mS/m, 50 mS/m, 10 mS/m or less;
v) a dry bulk density value of 50 g/L or more, for example, 80 g/L, 100 g/L, 150 g/L, 200 g/L, 250 g/L, 300 g/L, 400 g/L, 500 g/L, 600 g/L or more;
vi) a lignin content of 40% or more; for example, 50%, 60%, 70%, 80%, 90% or more; and
vii) an air-filled porosity value of less than 40%, for example, 30%, 27.9%, 25%, 20%, 10%, 5% or less.

34. The method according to claim 33 wherein the plant growth medium exhibits the following properties:
i) no detectable decomposition;
ii) a moisture retention of 60-75% at 0.1 bar;
iii) pH 4.43;
iv) an electrical conductivity of 67 mS/m;
v) a dry bulk density value of 50-110 g/L, preferably 80-110 g/L;
vi) a lignin content of 40%;
vii) an air-filled porosity value of 10-30%.

35. The method according to claim 1 wherein the plant growth material is a peat-substitute material.

36. A plant growth medium obtained or obtainable by the method of claim 1.

37. A peat-substitute material comprising or consisting of a plant growth medium according to claim 36.

38-39. (canceled)

40. A kit for performing a method according to claim 1 comprising one or more of the following:
a) a vessel for subjecting bioorganic matter to conditions capable of melting and/or hydrolysing and/or solubilising lignocellulose in the bioorganic matter, such as steam explosion apparatus;
b) a vessel for forming bio-alcohol from one or more sugar by fermentation;
c) bioorganic matter comprising lignocellulose;
d) one or more microbial agent capable of forming bio-alcohol from one or more sugar by fermentation; and
e) instructions for performing the method.
